SEQUENCE LISTING (26 sequences)

SEQ ID NO: 1 - protein, 116 aa, Artificial Sequence [sequence omitted]

SEQ ID NO: 2 - protein, 12 aa, Artificial Sequence
  Description of Artificial Sequence Synthetic peptide
GDGSSGGSGGAS

SEQ ID NO: 3 - protein, 108 aa, Artificial Sequence [sequence omitted]

SEQ ID NO: 4 - protein, 236 aa, Artificial Sequence
  Description of Artificial Sequence Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 5 - protein, 5 aa, Artificial Sequence
  Description of Artificial Sequence Synthetic peptide
SFSMS

SEQ ID NO: 6 - protein, 17 aa, Artificial Sequence
  Description of Artificial Sequence Synthetic peptide
SISGSSGTTYYADSVKG

SEQ ID NO: 7 - protein, 7 aa, Artificial Sequence
  Description of Artificial Sequence Synthetic peptide
PFPYFDY

SEQ ID NO: 8 - protein, 12 aa, Artificial Sequence
  Description of Artificial Sequence Synthetic peptide
RASQSVSSSFLA

SEQ ID NO: 9 - protein, 7 aa, Artificial Sequence
  Description of Artificial Sequence Synthetic peptide
YASSRAT

SEQ ID NO: 10 - protein, 9 aa, Artificial Sequence
  Description of Artificial Sequence Synthetic peptide
QQTGRIPPT

SEQ ID NO: 11 - protein, 118 aa, Artificial Sequence
  Description of Artificial Sequence Synthetic polypeptide
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 12 - protein, 5 aa, Artificial Sequence
  Description of Artificial Sequence Synthetic peptide
GGSGG

SEQ ID NO: 13 - protein, 108 aa, Artificial Sequence
  Description of Artificial Sequence Synthetic polypeptide
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 14 - protein, 231 aa, Artificial Sequence
  Description of Artificial Sequence Synthetic polypeptide
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 15 - protein, 3 aa, Artificial Sequence
  Description of Artificial Sequence Synthetic peptide
LFT

SEQ ID NO: 16 - protein, 6 aa, Artificial Sequence
  Description of Artificial Sequence Synthetic peptide
SGSGGS

SEQ ID NO: 17 - protein, 6 aa, Artificial Sequence
  Description of Artificial Sequence Synthetic peptide
STHLYL

SEQ ID NO: 18 - protein, 3 aa, Artificial Sequence
  Description of Artificial Sequence Synthetic peptide
MPF

SEQ ID NO: 19 - protein, 7 aa, Artificial Sequence
  Description of Artificial Sequence Synthetic peptide
GASSRAT

SEQ ID NO: 20 - protein, 6 aa, Artificial Sequence
  Description of Artificial Sequence Synthetic peptide
MRGRPP

SEQ ID NO: 21 - protein, 133 aa, Homo sapiens [sequence omitted]

SEQ ID NO: 22 - protein, 386 aa, Artificial Sequence
  Description of Artificial Sequence Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKEFSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 17 aa, Artificial Sequence
  Description of Artificial Sequence Synthetic peptide
EFSSSSGSSSSGSSSSG

SEQ ID NO: 24 - protein, 379 aa, Artificial Sequence
  Description of Artificial Sequence Synthetic polypeptide
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 25 - protein, 15 aa, Artificial Sequence
  Description of Artificial Sequence Synthetic peptide
SSSSGSSSSGSSSSG

SEQ ID NO: 26 - protein, 4 aa, Artificial Sequence
  Description of Artificial Sequence Synthetic peptide
DRDC

(19) United States
(12) Patent Application Publication
(10) Pub. No.: US 2019/0375837 A1
(43) Pub. Date: Dec. 12, 2019

(54) IMMUNOCYTOKINE COMBINATION THERAPY

(71) Applicant: PHILOGEN SPA, Siena (IT)

(72) Inventors: Dario NERI, Buchs (CH); Samuele CAZZAMALLI, Zurich (CH); Sarah WULHFARD, Baden (CH); Francesca PRETTO, Zurich (CH)

(73) Assignee: PHILOGEN SPA, Siena (IT)

(21) Appl. No.: 16/488,459

(22) PCT Filed: Feb. 23, 2018
(86) PCT No.: PCT/IB2018/051158

§ 371 Date: Aug. 23, 2019

Related U.S. Application Data
(60) Provisional application No. 62/463,162, filed on Feb. 24, 2017.
(60) Provisional application No. 62/506,137, filed on May 15, 2017.
(60) Provisional application No. 62/592,062, filed on Nov. 29, 2017.

(51) Int. Cl. C07K 16/24; A61K 38/20; A61K 31/427; A61K 47/54; A61K 47/65; A61K 47/68; A61P 35/00

(52) U.S. Cl. CPC C07K 16/246; A61K 38/2013; A61K 31/427; A61P 35/00; A61K 47/65; A61K 47/6813; A61K 47/545

(57) ABSTRACT

This invention relates to methods and compositions, in a combination therapy, for treatment of neoplastic disease, including tumors and cancer, wherein an immunocytokine and a small molecule drug conjugate which comprises a moiety capable of binding to a tumor-associated target, e.g., capable of binding to carbonic anhydrase IX (CAIX), are administered. In preferred embodiments, the immunocytokine comprises an antibody targeting the ED-B or ED-A domain of fibronectin and interleukin-2, and the small molecule drug conjugate comprises a ligand moiety capable of binding to CAIX, a linker, and a cytotoxic drug.

### SEQUENCE LISTING

The instant application contains a Sequence Listing which has been submitted electronically in ASCII format and is hereby incorporated by reference in its entirety. Said ASCII copy, created on Feb. 19, 2018, is named PHILO-0015-WO_SL.txt and is 20,811 bytes in size.

### FIELD OF THE INVENTION

This invention relates to a combination therapy for treatment of neoplastic disease, including tumors and cancer, wherein an immunocytokine and a small molecule drug conjugate which comprises a moiety capable of binding to a tumor-associated target, e.g., capable of binding to carbonic anhydrase IX (CAIX), are administered.

### BACKGROUND OF THE INVENTION

Despite many advances in the treatment of tumors and cancer, there remains a need for more effective therapies which are safer, more effective, specifically-targeted, which fully eradicate the cancer, and which overcome the limitations of prior treatments. Immunotherapies are particular promising avenue for research, but issues with efficacy, specificity and toxicity, as well as limitations in long-term results, remain.

### Immunocytokines

Cytokines are key mediators of innate and adaptive immunity. Many cytokines have been used for therapeutic purposes in patients with advanced cancer, but their administration is typically associated with severe toxicity, hampering dose escalation to therapeutically active regimens and their development as anticancer drugs.

To overcome these problems, the use of “immunocytokines” (i.e., cytokines fused to antibodies or antibody fragments) has been proposed, with the aim to concentrate the immune-system stimulating activity at the site of disease while sparing normal tissues.

A comprehensive review on immunocytokines has been published by Pasche & Neri, Drug Discovery Today, (2012), 17, 583-590.

### Interleukin 2 for Treatment of Cancer

Interleukin-2 (IL-2) is a type of cytokine signaling molecule in the immune system. It is a protein that regulates the activities of lymphocytes that are responsible for immunity. IL-2 is part of the body's natural response to microbial infection, and in discriminating between foreign (“non-self”) and “self”. IL-2 mediates its effects by binding to IL-2 receptors, which are expressed by lymphocytes.

Although high-dose IL-2, has been used in the treatment of renal cell carcinoma and melanoma, it is a highly toxic agent. IL-2 toxicity can manifest in multiple organ systems, most significantly the heart, lungs, kidneys, and central nervous system. The most common manifestation of IL-2 toxicity is capillary leak syndrome, resulting in a hypovolemic state and fluid accumulation in the extravascular space. Capillary leak syndrome can contribute significantly to development of oliguria, ischemia, and confusion. Hence there remains a need to improve the therapeutic window for IL-2, e.g., by conjugation with tumor-targeting antibodies.

### L19-IL2: An Immunocytokine for the Treatment of Cancer

WO2001/062298 reported for the first time the generation of an immunocytokine in which the L19 antibody—an anti-EDB antibody first disclosed in WO1999/058570 was conjugated to IL-2 to generate an immunoconjugate, referenced herein by the term immunocytokine (specifically, “L19-IL2”).

Subsequently, L19-IL2 was shown to have efficacy in different treatment modalities, including when used in combination with gemcitabine (disclosed in WO2007/115837), in combination with anti-CD20 antibodies (disclosed in WO2009/089858), with anti-CTLA-4 antibodies (disclosed in WO2013/010749), and with a conjugate of L19 and tumor necrosis factor (L19-TNF) (disclosed in WO2013/045125).

### Other IL-2-Based Immunocytokines

Other IL-2 based immunocytokines include F8-IL2 wherein an anti-ED-A antibody “F8” is conjugated to IL-2 and is used in combination with Sutent (disclosed in WO2010/078945) and in combination with an antibody-drug conjugate that targets the drug to a particular tissue (disclosed in WO2014/174105), wherein the combination of F8-IL2 and an antibody-drug conjugate (a targeted cytotoxic) for the treatment of Acute Myeloid Leukemia xenografted in mice was reported. Tumor eradication was observed in 4/5 mice.

### Small Molecule Drug Conjugates (SMDCs)

The use of cytotoxic agents is the basis of the treatment of cancer and other pathological conditions. Ideally, cytotoxic agents should specifically accumulate at site of disease, sparing normal tissues. In reality this does not happen at all, or very well. Many anticancer drugs do not preferentially accumulate in solid tumors. Indeed, it has been demonstrated in tumor-bearing mice that only a minimal portion of the injected drug reaches the neoplastic mass in comparison to the amount of cytotoxic agent that reaches healthy organs. More importantly, emerging Positron Emission Tomography (PET) studies, performed with radiolabeled cytotoxic drugs (e.g., ¹¹C-docetaxel) have unequivocally shown that these toxic agents do not preferentially accumulate on neoplastic lesions, but rather target other structures in the body (e.g., clearance-associated organs) (Van der Veldt et al., Eur. J. Nucl. Med. Mol. Imaging, 2010, 37: 1950; Van der Veldt et al., Clin. Cancer Res., 2013, 19: 4163).

The targeted delivery of highly potent cytotoxic agents into diseased tissues is therefore desirable for the treatment of cancer and other serious conditions. By attaching a therapeutic effector through a cleavable linker to a ligand specific to a marker of disease, the effector preferentially accumulates and acts at the intended site of action, thus increasing the effectively applied dose while reducing side effects. To date, monoclonal antibodies capable of selective internalization into the target tumor cells have been considered as the ligands of choice and, indeed, research in the field of antibody-drug conjugates (ADCs) has led to the recent approval of two ADCs for applications in oncology: brentuximab vedotin and trastuzumab emtansine (Gualberto A., Expert Opin Investig Drugs, 2012, 21: 205-16; Beck A and Reichert J M, MAbs, 2014, 6: 15-7).

However, antibodies are large macromolecules and thus often have difficulties penetrating deeply into solid tumors. In addition, they can be immunogenic and typically long circulation times can lead to premature drug release and undesired side effects. Moreover, the production of ADCs is expensive, reflecting the need for clinical-grade manufacturing of antibodies, drugs and the resulting conjugates.

The use of smaller ligands as delivery vehicles such as peptides or small drug-like molecules capable of selective internalization into tumor cells could potentially overcome some of the above-mentioned problems. Their reduced size should aid in tissue penetration, they should be non-immunogenic and amenable to classic organic synthesis thus reducing manufacturing costs. The favorable properties of drug conjugates using folic acid or ligands against prostate-specific membrane antigen (PSMA) as delivery vehicles have been demonstrated and a folate conjugate has recently entered Phase III clinical studies. However, only a few such conjugates have been successfully identified.

### SMDC's Specific for Carbonic Anhydrase (IX)

WO2015/114171 reports the generation of a small organic molecule capable of high-affinity binding to Carbonic Anhydrase IX, an antigen that is over-expressed in renal cell carcinoma. When such binding molecule is armed with suitable linker and cytotoxic drug payload, successful treatment of cancer is shown.

Need for Improved Therapeutics in Kidney Cancer with IL-2-Based Immunocytokines

In a clinical trial featuring the administration of L19-IL2 in 18 patients with renal cell carcinoma, Johanssen et al. (European Journal of Cancer, 2010, 46: 2926-2935) reported no objective responses (page 2934, first column).

WO2010/078945 reported efficacy of a combination for F8-IL2 and sunitinib in a mouse model of clear cell renal cell carcinoma (Caki-1). While tumor shrinkage is observed, no complete remission was seen.

### Need for Improved Therapeutics in Kidney Cancer Using CAIX-Binder-Based Small Molecule Drug Conjugates (SMDC)

Krall et al. (Chem. Sci. (2014) 5: 3640-3644) reported complete tumor regression in 2 out of 6 mice in a mouse model xenografted with SKRC-52, a metastatic human renal carcinoma cell line, using a CAIX-binder conjugated with DM-1 cytotoxic drug.

Cazzamalli et al. (Mol Cancer Ther., (2016) 15(12): 2926-2935) reported antitumor activity in nude mice bearing xenografted SKRC-52 renal cell carcinomas of a CAIX-binder conjugated with cytotoxic drugs MMAE or PNU-159682, using a dipeptide linker (valine-citrulline). Only one mouse out of five experienced a complete remission (FIG. 6).

Cazzamalli et al. (J Control Release (2017) 246: 39-45) tested a CAIX-binder conjugated with cytotoxic drug MMAE using various dipeptide linkers in nude mice bearing xenografted SKRC-52 renal cell carcinomas. They report that SMDCs having the dipeptide linkers valine-citrulline and valine-alanine linkers showed better serum stability and a superior therapeutic activity compared to the SMDCs with valine-lysine and valine-arginine linkers. However they do not show any complete tumor remission (FIG. 4) of tumors induced by SKRC-52.

Thus it is clear that there remains a need for better immunotherapeutic cancer treatments, including optimizing the use of immunocytokines and targeted cytotoxic agents that can overcome the difficulties encountered with targeting moieties such as antibodies, and even more particularly for the treatment of kidney cancer and other CAIX-expressing cancers such as colorectal, urothelial, lung, stomach, pancreas, breast, head and neck and cervical cancer.

### SUMMARY OF THE INVENTION

Reported here are unexpected effects on neoplastic disease, specifically tumors, resulting from administration of a combination of an immunocytokine and a small molecule drug conjugate, which comprises a moiety capable of binding to a tumor-associated target.

Thus, in one aspect of the invention, a method for the treatment of cancer is provided, comprising the administration of a therapeutic combination comprising:

(i) an immunocytokine, and

(ii) a small molecule drug conjugate (SMDC).

In another aspect of the invention, a therapeutic combination or a therapeutic composition comprising:

(i) an immunocytokine, and

(ii) a small molecule drug conjugate (SMDC), for use in the treatment of cancer is provided.

In one embodiment, the immunocytokine comprises an antibody or antibody fragment conjugated to a cytokine, and the small molecule comprises a moiety capable of binding to carbonic anhydrase IX (CAIX).

In a preferred embodiment, the immunocytokine comprises:
(a) an antibody targeting the ED-B domain of fibronectin, and
(b) interleukin-2.

In another preferred embodiment, the immunocytokine comprises:
(a) an antibody targeting the ED-A domain of fibronectin, and
(b) interleukin-2.

In another preferred embodiment, the small molecule drug conjugate comprises:
(a) a moiety capable of binding to CAIX,
(b) a linker, and
(c) a cytotoxic drug.

In another preferred embodiment, the antibody is L19.

In another preferred embodiment, the antibody is F8.

In a still further preferred embodiment, the immunocytokine comprises L19-IL2.

In a still further preferred embodiment, the immunocytokine comprises F8-IL2.

In one embodiment, the neoplastic disease is cancer.

In one embodiment, the cancer is a CAIX-expressing cancer.

In a preferred embodiment, the cancer is a kidney cancer.

In another preferred embodiment, the cancer is a colorectal cancer.

In additional aspects of the invention, compositions, pharmaceutical compositions, and kits comprising compositions for administration to patients in need of treatment are provided.

In another aspect of the invention, a composition comprising
(i) an immunocytokine and
(ii) a small molecule drug conjugate (SMDC),
   wherein the immunocytokine comprises an antibody or antibody fragment conjugated to a cytokine, and the small molecule comprises a moiety capable of binding to a tumor-associated target is provided.

In yet another aspect of the invention, use of a composition comprising
(i) an immunocytokine and
(ii) a small molecule drug conjugate (SMDC),
   wherein

the immunocytokine comprises an antibody or antibody fragment conjugated to a cytokine, and

the small molecule comprises a moiety capable of binding to a tumor-associated target in the manufacture of a medicament for the treatment of a neoplastic disease, is provided.

### BRIEF DESCRIPTION OF THE DRAWINGS

FIGS. 1A and 1B disclose analytical Reversed-Phase Ultra Performance Liquid Chromatography (UPLC) traces of compounds 1 and 2 during synthesis. FIG. 1A discloses analytical UPLC trace of compound 1 on a BEH (Ethylene-Bridged-Hybrid) C18 Column, 130 Å, 1.7 μm, 2.1 mm×50 mm at a flow rate of 0.6 ml min⁻¹, 5% MeCN (acetonitrile) in 0.1% aq. FA to 80% MeCN in 6 min. FIG. 1B discloses Analytical UPLC trace of compound 2 on a BEH C18 Column, 130 Å, 1.7 μm, 2.1 mm×50 mm at a flow rate of 0.6 ml min⁻¹, 5% MeCN in 0.1% aq. FA to 80% MeCN in 6 min.

FIGS. 2A and 2B disclose the therapeutic activity of L19-IL2 immunocytokine in combination with CAIX targeted SMDCs in human renal cell carcinoma (RCC) xenografts. BALB/c nude mice bearing established (50-100 mg subcutaneous) SKRC-52 RCC were treated with AAZ*-ValCit-MMAE (compound 2; 250 nmol/kg; IV; black arrow) and L19-IL2 (2.5 mg/Kg; IV; grey arrow), as single therapy or in combination regimens. The targeted chemotherapeutic agent was administered 24 hours before the immunocytokine. FIG. 2A compares the therapeutic activity of the different treatments. In FIG. 2B, percentage changes of body weight during the experiment are represented. Statistical analysis was performed by 2-way ANOVA test followed by Bonferroni post-test.

SMDCs=Small Molecule Drug Conjugates

N=5 mice per group.

CR=complete tumor regression.

**** indicates p<0.0001;

*** indicates p<0.001;

** indicates p<0.01.

FIG. 3 depicts photographs of BALB/c nude mice bearing subcutaneous SKRC-52 tumors, on day 17 after tumor implantation, after having received different treatments. Potent antitumor activity was observed for L19-IL2 and AAZ*-ValCit-MMAE (compound 2), but only the combination of the two agents led to the complete regression of the tumor in the model.

FIGS. 4A and 4B show a tumor rechallenge experiment and the therapeutic activity of compound 2 and of L19-IL2 on regrowing tumors. Mice cured by the combination treatment of L19-IL2 with SMDC compound 2 (see FIG. 2A) were rechallenged with the original tumor SKRC-52 renal cell carcinoma by subcutaneous injection of tumor cell suspension, 20 days after they were cured from the primary tumor. All the tumors injected were growing. Mice were rearranged in two groups (2 and 3 animals) and treated with either AAZ*-ValCit-MMAE (compound 2; 250 nmol/kg; IV; black arrow; 2 mice), or with L19-IL2 (2.5 mg/Kg; IV; grey arrow; 3 mice). FIG. 4A compares the therapeutic activity of the different treatments and shows mean tumor volumes over the time frame of the experiment. Only the treatment with the immunocytokine caused a second complete tumor regression for all 3 animals composing treatment group. In FIG. 4B, body weight changes are shown. Statistical analysis was performed by 2-way ANOVA test followed by Bonferroni post-test.

N=3 animals per group.

CR=complete tumor regression.

**** indicates p<0.0001.

FIGS. 5A and 5B disclose the therapeutic activity of F8-IL2 immunocytokine in combination with CAIX targeted SMDCs in human renal cell carcinoma (RCC) xenografts. BALB/c nude mice bearing established (50-100 mg subcutaneous) SKRC-52 RCC were treated with AAZ*-ValCit-MMAE (compound 2; 250 nmol/kg; IV; black arrow) and F8-IL2 (2.5 mg/Kg; IV; grey arrow) in combination regimens. As control, a second group of mice were treated with F8-IL2 only (2.5 mg/Kg; IV). The targeted chemotherapeutic agent was administered with a 24 hours interval before the immunocytokine. FIG. 5A compares the therapeutic activity of the different treatment. In FIG. 5B percentage changes of body weight during the experiment are represented. A better control of the tumor growth was achieved compared to the control. Statistical analysis was performed by 2-way ANOVA test followed by Bonferroni post-test.

SMDCs=Small Molecule Drug Conjugates.

N=3 mice per group.

FIGS. 6A-6C show the in vitro and in vivo evaluation of the new murine colorectal carcinoma model CT26.3E10 transfected with the human antigen CAIX. FIG. 6A depicts the cloning scheme of human CAIX in pcDNA3.1(+). In FIG. 6B., the results of a FACS analysis for expression of human CAIX on SKRC-52, CT26.wt and transfected CT26.3E10 cells are shown Staining was performed with a human anti-CAIX specific antibody and the corresponding signal was amplified with an anti-human AlexaFluor488 secondary antibody. In FIG. 6C, the in vivo targeting performance of AAZ-IRdye680RD (compound 3) in immunocompetent BALB/c mice bearing CAIX transfected CT26.3E10 tumors was evaluated by near-infrared fluorescence imaging. Selective tumor uptake at early time points (3 and 6 hours) was observed, as compared to the biodistribution of the molecule in CAIX-negative CT26.wt tumor bearing mice.

FIGS. 7A and 7B show the therapeutic activity of AAZ*-ValCit-MMAE in combination with the immunocytokine L19-IL2 in BALB/c mice bearing CT26.3E10 colorectal carcinoma. BALB/c mice bearing established subcutaneous, human CAIX expressing tumors were treated with AAZ*-ValCit-MMAE (compound 2; 250 nmol/kg; IV; black arrow) and L19-IL2 (2.5 mg/Kg; IV; white arrow), as monotherapy or in a combination regimen. FIG. 7A compares the therapeutic activity of the different treatments. Data points represent mean tumor volume ±SEM, n=5 per group. CR=Complete Responses. In FIG. 7B, the percentage changes of body weight during the experiment are shown. **** indicates p<0.0001; *** indicates p<0.001; ** indicates p<0.01 (2-way ANOVA test, followed by Bonferroni post-test).

FIGS. 8A and 8B disclose analytical Reversed-Phase Ultra Performance Liquid Chromatography (UPLC) traces of compounds 5 and 6 during synthesis and chemical structures of CAIX-targeting SMDCs. FIG. 8A discloses analytical UPLC trace of compound 5 on a BEH C18 Column, 130 Å, 1.7 μm, 2.1 mm×50 mm at a flow rate of 0.6 ml min-1, 5% MeCN in 0.1% aq. FA to 80% MeCN in 6 min. FIG. 8B Analytical UPLC trace of compound 6 on a BEH C18 Column, 130 Å, 1.7 μm, 2.1 mm×50 mm at a flow rate of 0.6 ml min-1, 5% MeCN in 0.1% aq. FA to 80% MeCN in 6 min FIG. 8C shows the chemical structures of CAIX-targeting SMDCs. Compounds display an acetazolamide targeting ligand (AAZ; compound 4), an affinity matured version of acetazolamide (AAZ*; compound 2) or an amide (serving as negative control; compound 6). All products feature the cytotoxic payload MMAE, an Asp-Arg-Asp-Cys (SEQ ID NO: 26) peptide spacer, a spacer unit, a Valine-Citrulline (ValCit) dipeptide cleavable linker and a p-amino benzyl (PAB) self immolative spacer (linker).

FIGS. 9A and 9B disclose a comparison of the in vivo efficacy of AAZ*-ValCit-MMAE and AAZ-ValCit-MMAE (compounds 2 and 4) in BALB/c nu/nu mice bearing subcutaneous SKRC-52 renal cell carcinomas. Cytotoxic derivative (compound 6) devoid of the acetazolamide moiety was used as negative control. All the compounds were injected intravenously at the dose of 250 nmol/Kg per administration. The “presaturation” group was treated with a 50-fold dose of AAZ* ligand (1.25 μmol/Kg; compound 7) directly followed by an administration of compound 2 (250 nmol/Kg). FIG. 9A compares the therapeutic activity of the different treatment. Data points represent mean tumor volume ±SEM, n=4 per group. SMDC 2 based on the affinity matured AAZ* ligand exhibited a superior antitumor activity when compared with SMDC 4, based on the non-matured AAZ targeting moiety. The therapeutic efficacy of SMDC 2 was reduced significantly by the presaturation with an excess of free AAZ*. FIG. 9B shows the percentage changes of body weight during the experiment are represented. **** indicates p<0.0001; ** indicates p<0.01; * indicates p<0.05; ns indicates p>0.05 (2-way ANOVA test, followed by Bonferroni post-test).

FIGS. 10A and 10B disclose analytical Reversed-Phase Ultra Performance Liquid Chromatography (UPLC) traces of compound 8 during synthesis and the quantitative biodistribution of AAZ*-⁹⁹mTc (compound 8) and of AAZ-⁹⁹mTc (compound 9) in the SKRC-52 model. FIG. 10A discloses analytical HPLC trace of compound 8 on a on a Synergi RP Polar column a t a flow rate of 4 ml min-1, 5% MeCN in 0.1% aq. TFA to 80% MeCN in 20 min. The injection peak at around 2 minutes is an artefact. FIG. 10B shows chemical structures and organ distribution of ⁹⁹mTc-radiolabeled AAZ and AAZ* (compounds 9 and 8, respectively) in BALB/c nu/nu mice bearing SKRC-52 xenografts (n=3 per group). Compound 10, devoid of the anti-CAIX targeting moiety, served as negative control for the experiment. The data, expressed as mean % Injected Dose/gram of tissue ±SD (% ID/gram), correspond to the 6 hours time point after the intravenous administration of the radiolabeled compound.

FIG. 11 shows chemical structures and qualitative biodistribution of AAZ*-IRdye680RD (compound 11) and of the corresponding negative control (compound 12), devoid of the acetazolamide-targeting moiety. A selective tumor uptake of AAZ*-IRdye680RD can be observed at early time points (1, 3 and 6 hours) in immunodeficient BALB/c nu/nu mice bearing SKRC-52 tumors (white arrows) by near-infrared fluorescence imaging.

### DETAILED DESCRIPTION OF THE INVENTION

Unless otherwise stated, all technical and scientific terms used herein have the same meanings as commonly understood by one of ordinary skill in the art to which this invention belongs. Any methods and materials with similar or equivalent function to those described herein can be used in the practice or testing of the present invention. Methods, devices, and materials suitable for such uses are now described. The entire disclosures of all applications, patents and publications cited herein are expressly incorporated herein by reference in their entirety, in particular for the purpose of describing and disclosing the methodologies, reagents, and tools reported in the publications that might be used in connection with the invention.

The methods and techniques of the present application are generally performed according to conventional methods well known to those of skill in the art and as described in various general and more specific references that are cited and discussed throughout the present specification unless otherwise indicated. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer-Verlag.

### Definitions

An “immunocytokine” according to the invention comprises a cytokine linked to an antibody molecule, which targets the cytokine to the site of the tumor. In one embodiment, the antibody molecule binds a splice isoform of an extracellular matrix component, which is selectively expressed by the extracellular matrix in tumor tissue. By combining this targeting effect with direct administration of the immunocytokine to the tumor site, a very localized administration is achieved, which concentrates the effect of the cytokine at the tumor site and reduces side effects and toxicity associated with systemic use of a cytokine.

Definitions of the elements of the immunocytokine can be found in various references cited herein, including, e.g., in WO2013045125, which definitions are incorporated by reference herein, including the definitions interchangeably referencing these molecules as “immunoconjugates.”

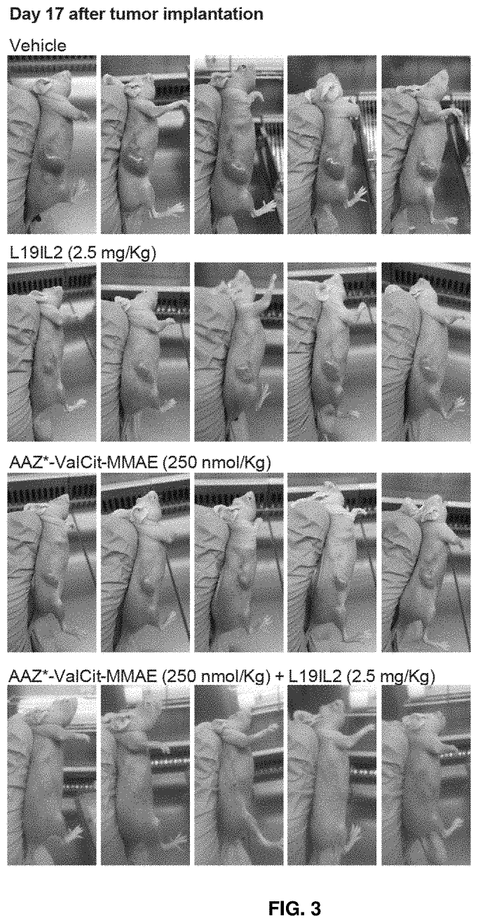

A number of splice isoforms of tumor extracellular matrix components are known, and antibody molecules targeting any such isoform may be used to selectively target the tumor. These include splice isoforms of fibronectin, such as B-FN. B-FN includes an extra domain ED-B, and antibody molecules of the invention are preferably targeted to this domain. A preferred antibody molecule comprises the complementarity determining regions (CDRs) of antibody L19, in particular Sequence ID Numbers (SIDs) 1-6, as illustrated in FIG. 3 of WO2013/045125 (corresponding to SEQ ID NOs 5-10 herein). Preferably, the antibody molecule of the IL2 immunocytokine comprises the L19 VH domain and/or the L19 VL domain Amino acid sequences of the L19 VH and VL domains are SID:7 and SID:9 respectively, as illustrated in FIG. 3 of WO2013/045125 (corresponding to SEQ ID NO: 1 and SEQ ID NO: 3, respectively, herein).

Antibodies which bind the ED-A of human fibronectin, and thus also human A-FN, are known in the art and include antibody F8. An antibody molecule for use in the invention preferably has the CDRs of antibody F8 set forth in SEQ ID NOs 15-20. More preferably, an antibody for use in the invention comprises the VH and/or VL domains of antibody F8 set forth in SEQ ID NO: 11 and SEQ ID NO: 13, respectively. Yet more preferably, an antibody for use in the invention comprises the VH and VL domains of antibody F8 set forth in SEQ ID NO: 11 and SEQ ID NO: 13. The F8 antibody is preferably in diabody or scFv format, most preferably in diabody format. Where the F8 antibody is in diabody format, the antibody molecule for use in the invention preferably has the amino acid sequence set forth in SEQ ID NO: 14.

An antibody molecule for use in the invention may bind the A-FN and/or the ED-A of fibronectin, with the same affinity as anti-ED-A antibody F8, e.g., in diabody format, or with a higher affinity.

The immunocytokine preferably comprises an antibody or antibody fragment conjugated to a cytokine. The antibody fragment can be any suitable antigen-binding fragment of an immunoglobulin, such as a Fab, F(ab)₂, Fv, scFv, diabody, dAb, a Vhh domain, or any other immunoglobulin-based binding domain. Alternatives can be based on non-immunoglobulin scaffolds, and are known in the art. Further possibilities include peptides and nucleic acid aptamers. In a preferred embodiment, the antibody fragment is an ScFv, diabody or a small immune protein (SIP).

Preferably the antibody molecule is a single chain Fv (scFv) or other antibody fragment of low molecular weight and/or lacking an Fc region. These properties assist with targeting and tissue penetration of the immunocytokine at the tumor site. A preferred antibody molecule is scFv-L19, which is an scFv comprising an L19 VH domain and an L19 VL domain, wherein the VH and VL are conjoined in a single polypeptide chain by a peptide linker sequence. The VH domain contains VH CDR1, CDR2 and CDR3 sequences, and the VL domain contains VL CDR1, CDR2 and CDR3 sequences. The VH domain may have an amino acid sequence as disclosed as SID:7 (SEQ ID NO: 1 herein) as illustrated in FIG. 3 of WO2013/045125. The VL domain may have an amino acid sequence as disclosed in SID:9 (SEQ ID NO: 3 herein) as illustrated in FIG. 3 of WO2013/045125. The VH and VL domains can be joined by a peptide linker such as the 12 residue linker as disclosed as SID:8 as illustrated in FIG. 3 in WO2013/045125 (corresponding to SEQ ID NO: 2 herein). Preferably, the scFv-L19 comprises or consists of the amino acid sequence disclosed as SID:10 as illustrated in FIG. 3 (corresponding to SEQ ID NO: 4 herein).

A preferred antibody molecule is the diabody F8, which comprises two scFv molecules. Where the antibody molecule is a diabody the VH and VL domains are preferably linked to a 5 to 12 amino acid linker. A diabody comprises two VH-VL molecules which associate to form a dimer. The VH domain contains VH CDR1, CDR2 and CDR3 sequences, and the VL domain contains VL CDR1, CDR2 and CDR3 sequences. The VH domain may have an amino acid sequence as disclosed as SEQ ID NO: 11. The VL domain may have an amino acid sequence as disclosed in SEQ ID NO: 13. The VH and VL domains of each VH-VL molecule are preferably linked by a 5 to 12 amino acid linker. For example, the VH and VL domains may be linked by an amino acid linker which is 5, 6, 7, 8, 9, 10, 11, 12 amino acid in length. Preferably, the amino acid linker is 5 amino acids in length. Suitable linkers are known in the art and include the linker sequence set forth in SEQ ID NO: 12. Preferably, the diabody F8 comprises or consists of the amino acid sequence disclosed as SEQ ID NO: 14.

In a preferred embodiment, the cytokine is an interleukin, such as IL-2 or IL-12, or TNF-α. Preferably the cytokine is IL2, more preferably IL2 is human IL2.

The IL2 preferably comprises or consist of the sequence set forth in SEQ ID NO: 21. Typically, IL2 has at least 70%, more preferably one of at least 75%, 80%, 85%, 90%, 95%, 96%, 97%, 98%, 99% or 100%, sequence identity to the amino acid sequence set forth in SEQ ID NO: 21. IL2 in the immunocytokine of the invention retains a biological activity of human IL2, e.g., the ability to inhibit cell proliferation.

The immunocytokine of the present invention may comprise or consist of the sequence shown in SEQ ID NO: 22. In this embodiment, the immunocytokine may have at least 70%, more preferably one of at least 75%, 80%, 85%, 90%, 95%, 96%, 97%, 98%, 99% or 100%, sequence identity to the amino acid sequence shown in SEQ ID NO: 22.

Alternatively, the immunocytokine of the present invention may comprise or consist of the sequence shown in SEQ ID NO: 24. In this embodiment, the immunocytokine may have at least 70%, more preferably one of at least 75%, 80%, 85%, 90%, 95%, 96%, 97%, 98%, 99% or 100%, sequence identity to the amino acid sequence shown in SEQ ID NO: 24. A molecular linker such as a peptide may be used to join the cytokine to the antibody molecule, facilitating expression of all or part of the immunocytokine as a fusion protein. Where the antibody molecule is also a single chain molecule, such as scFv, the entire immunocytokine polypeptide chain may conveniently be produced as a fusion protein.

Preferably, the antibody molecule is connected to the cytokine through a linker, preferably an amino acid linker.

The amino acid linker connecting the antibody molecule and the cytokine may be a flexible amino acid linker. Suitable examples of amino acid linker sequences are known in the art. The linker may be 10-20 amino acids, preferably 11-17 amino acids in length. More preferably, the linker is 15-17 amino acids in length. In particularly preferred embodiments, the linker may have the sequence set forth in SEQ ID NO: 25, or the sequence set forth in SEQ ID NO: 23.

Optionally, the immunocytokine carries a detectable and/or functional label, such as a radioactive isotope. Radiolabeled L19, and its use in cancer therapy, has been described before (WO2003/076469). In a preferred embodiment, the immunocytokine is L19-IL2, e.g., as disclosed in various publications, including WO2001/062298, WO2007/115837, WO2009/089858, WO2013/010749 and WO2013/045125.

In a preferred embodiment, the immunocytokine is F8-IL2, e.g., as disclosed in various publications, including WO2010/078945, WO2011/015333, WO2014/173570, Wieckowski S, Hemmerle T, Prince SS, Schlienger B D, Hillinger S, Neri D, Zippelius A, “Therapeutic efficacy of the F8-IL2 immunocytokine in a metastatic mouse model of lung adenocarcinoma” Lung Cancer 2015 April; 88(1):9-15; Pretto F, Elia G, Castioni N, Neri D, “Preclinical evaluation of IL2-based immunocytokines supports their use in combination with dacarbazine, paclitaxel and TNF based immunotherapy” Cancer Immunol Immunother 2014 September; 63(9):901-10; and Moschetta M, Pretto F, Berndt A, Galler K, Richter P, Bassi A, Oliva P, Micotti E, Valbusa G, Schwager K, Kaspar M, Trachsel E, Kosmehl H, Bani M R, Neri D, Giavazzi R, “Paclitaxel enhances therapeutic efficacy of the F8-IL2immunocytokine to EDA-fibronectin-positive metastatic human melanoma xenografts” Cancer Res 2012 Apr. 1; 72(7):1814-24.

“Small molecule drug conjugates” according to the invention are targeted therapeutic agents which comprise a low molecular weight ligand for binding to a target tissue conjugated to a drug, in a preferred embodiment conjugated by a cleavable linker, for delivery of the drug to targeted tissues or cells. In one embodiment, the invention relates to the application of such SMDCs for the delivery of drugs that can kill or inhibit tumor cells for the treatment of tumors and/or cancer, in conjunction with immunocytokines.

In particular, the invention comprises, in addition to an immunocytokine, a targeted therapeutic agent comprising a compound of formula (I):

B-L-D (I),

wherein:

B is a low molecular weight binding moiety (ligand) for a tumor-associated target, preferably for a Carbonic Anhydrase;

D is a drug moiety; and

L is a linker group that undergoes cleavage in vivo for releasing said drug moiety in an active form, said linker optionally further comprising one or more spacers between the B and D moieties.

In a preferred embodiment according to the invention, the “binding moiety” (ligand B) suitably binds to a tumor-associated carbonic anhydrase enzyme, most preferably it binds to Carbonic Anhydrase IX (CAIX). The binding to the carbonic anhydrase is suitably selective or specific, whereby the binding moiety B accumulates in vivo at sites, such as tumors, where carbonic anhydrase is present at elevated levels. Alternatively or additionally, the binding moiety may bind to other carbonic anhydrases such as Carbonic Anhydrase XII.

Suitably, the SMDC compound of Formula (I) has a molecular weight less than about 8,000, more suitably less than about 5000, and most suitably less than about 2000. In contrast to antibodies, small molecules can diffuse out of blood vessels in a matter of seconds. The distribution is not restricted to perivascular space, but involves also deep penetration into tissues. This results in faster, deeper and more efficient drug targeting by the drug portion of the SMDCs of the invention.

Suitable exemplary SMDCs are set forth in WO2015114171, including the definitions of a low molecular weight binding moiety for a Carbonic Anhydrase B, the drug moiety D; and the linker group L that undergoes cleavage in vivo for releasing said drug moiety in an active form. In addition, WO2015114171 discloses optional cleavage agents for cleaving the linker L at a later time point following SMDC administration, in order to trigger an efficient release of the drug payload when suitable tumor:blood and tumor:organ ratios have been achieved.

Suitably, the molecular weight of the binding moiety (ligand) is less than about 10,000, preferably less than about 3000, most preferably less than about 1000. In some embodiments, the binding moiety (ligand) is a peptide. In other embodiments, the binding moiety (ligand) is not a peptide. In other embodiments, the binding moiety (ligand) consists in a chemical combination of peptidic and non-peptidic structures. The possibility of using small organic or inorganic molecules as ligands instead of antibodies allows those molecules to have complexity which is amenable to chemical synthesis. The core of the structures can vary from pure organic compounds to structures that are based on peptide scaffolds and even inorganic structures such as boron and other clusters.

The binding moiety may be based on a compound that is known to bind strongly to the target. Alternatively, the binding moiety may be identified by one or more known screening methods for identifying compounds that bind selectively to the target protein of interest.

For example, improved variants of the ligands described below, or new ligands for binding selectively to target proteins of interest, can be found by screening methods using modern medicinal chemistry technologies, e.g., DNA-encoded chemical library technologies as described in WO2009077173 and by R. E. Kleiner et al. in Chemical Society Reviews 40 5707-5717 (2011), L Mannocci et al. in Chemical Communications 47, 12747-12753 (2011) and S. Brenner et al. in Proceedings of the National Academy of Sciences of the USA 89 5381-5383 (1992). An example of a screening method used to identify the best binding moiety for CAIX from a library of 111,100 small organic molecules is described in more detail in WO2015114171.

The binding moiety (ligand) must tolerate attachment to the rest of the conjugate while maintaining binding affinity for its target. Suitably, the conjugate exhibits a binding affinity to its target (typically recombinant CAIX) such that the resulting complex has K_{D} less than about 50 nM, more suitably less than about 30 nM, less than about 20 nM, less than 10 nM, less than 5 nM, less than 2 nM, or less than 1 nM.

Carbonic anhydrases are thought to have a catalytic mechanism which relies upon an active site which contains a coordinated zinc ion. Carbonic anhydrase inhibitors such as acetazolamide and methazolamide which have terminal sulfonamido groups are thought to act by forming an adduct between the zinc ion and the terminal nitrogen of the sulfonamide. Accordingly, the binding moieties in the conjugates according to the present invention suitably have a terminal sulfonamide (—SO₂NH₂), sulfamate (—OSO₂NH₂) or sulfamide (—NHSO₂NH₂) group. Most suitably, the terminal group is a sulfonamide group. Suitably, the terminal sulfonamide, sulfamate or sulfamide group is bonded to an aryl group, for example to form an arylsulfonamido group —ArSO₂NH₂.

The aryl group in these embodiments typically has a single ring or two fused rings. The aryl group may be carbocyclic or heterocyclic and may be substituted or unsubstituted. Typically, small substituents are preferred such as Me, Et, OH, MeO, CF₃, F, Cl, Br, I and CN. Whether or not the Ar group is substituted, two ring positions are taken up with the terminal sulfonamide group and the bond to the rest of the conjugate. These two ring positions may be at any point on the Ar ring.

Suitably, the aryl group is a thiadiazolyl group. In these embodiments, the ligand suitably comprises the following terminal moiety (T1):

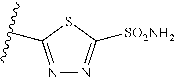 [SMILES: *c1nnc(S(N)(=O)=O)s1]

Suitably, the remainder of the conjugate is bonded to the thiadiazolyl group through an amide group, whereby the binding moiety (ligand) comprises the following terminal moiety having a structure similar to the terminal moiety of acetazolamide (T2):

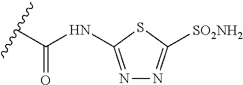 [SMILES: *C(=O)Nc1nnc(S(N)(=O)=O)s1]

In other embodiments, the above terminal moiety is modified by 4-N methylation of the thiadiazole group whereby the binding moiety (ligand) comprises the following terminal moiety having a structure similar to the terminal moiety of methazolamide (T3):

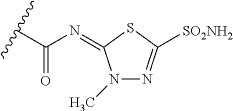 [SMILES: *C(=O)/N=c1/sc(S(N)(=O)=O)nn1C]

The binding moieties used in the present invention are not limited to sulfonamido derivatives. For example, coumarin ligands are also known to bind to CAIX. The skilled person using the techniques described herein and common general knowledge will be able to identify further suitable ligands for use as the binding moiety.

In embodiments, the binding moiety (ligand) B may be a univalent binding moiety or a multivalent binding moiety, for example a bivalent binding moiety. The term “univalent binding moiety” refers to a binding moiety comprising a single ligand for binding to CAIX. The term “multivalent binding moiety” refers to a binding moiety having two or more binding ligands (which may be the same or different) for binding to the target entity. Suitably, the binding moiety is bivalent. The two or more binding ligands are separated by suitable spacer groups on the multivalent binding moieties. The use of multivalent binding moieties can provide enhanced binding of the binding moiety to the target.

Suitably, in these embodiments at least one of the two or more binding ligands comprises a terminal moiety

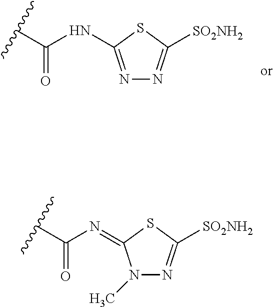 [SMILES: *C(=O)Nc1nnc(S(N)(=O)=O)s1 ; *C(=O)/N=c1/sc(S(N)(=O)=O)nn1C]

In these embodiments binding moiety (ligand) is suitably a bivalent binding moiety comprising a first binding ligand comprising a terminal moiety as defined above and a second binding ligand selected from the group consisting of ligands having a terminal moiety as defined above and ligands having the terminal group

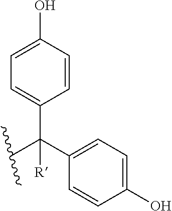

wherein R′ is H or C1-C7 alkyl, C1-C7 alkenyl, or C1-C7 heteroalkyl, optionally substituted with one, two or three substituents, and preferably R′ is methyl.

In other embodiments, the binding moiety (ligand) suitably comprises or consists essentially of:

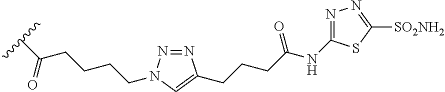 [SMILES: *C(=O)CCCCn1cc(CCCC(=O)Nc2nnc(S(N)(=O)=O)s2)nn1]

In other embodiments, the binding moiety (ligand) suitably comprises or consists essentially of:

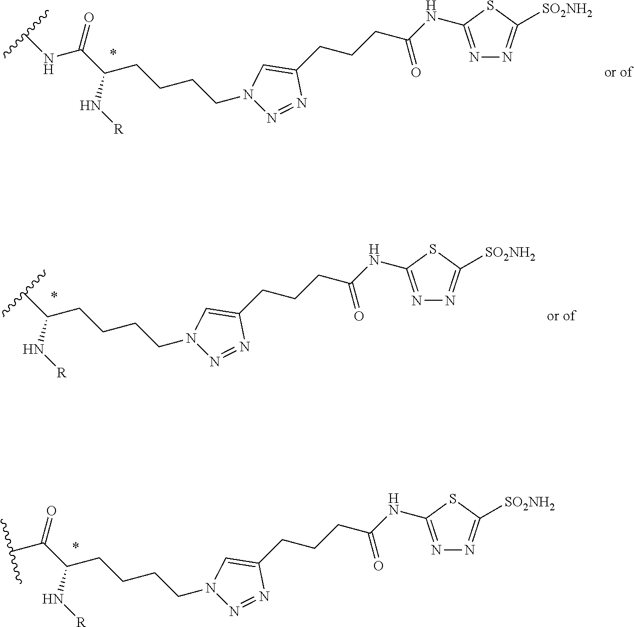

wherein the substituent R is selected from the group consisting of:

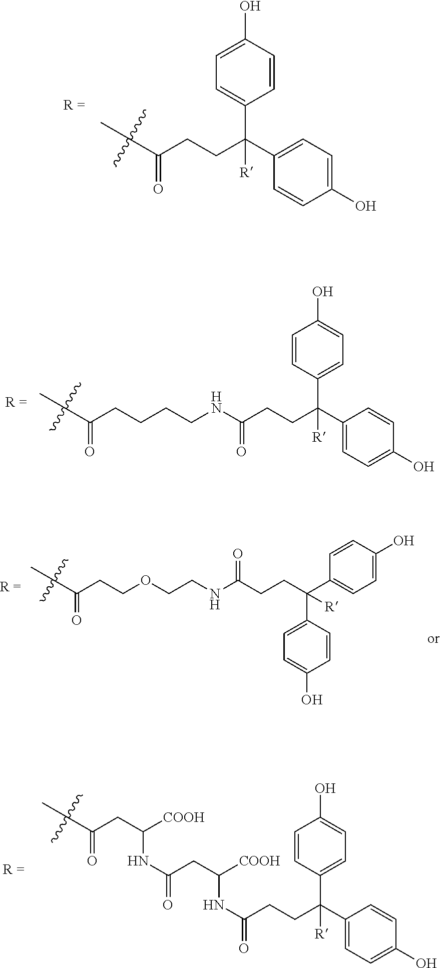

wherein R′ is H or C1-C7 alkyl, C1-C7 alkenyl, or C1-C7 heteroalkyl, optionally substituted with one, two or three substituents, and preferably R′ is methyl.

When there is an amine substituent of the a (alpha) carbon indicated with *, it is preferred that the chiral amine in the (S) configuration as described in WO2015/114171, although it may also be in the (R) configuration, e.g.:

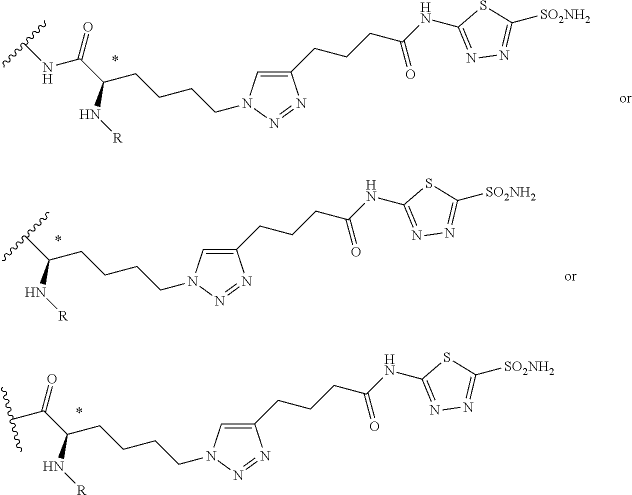

wherein the substituent R is selected from the group consisting of:

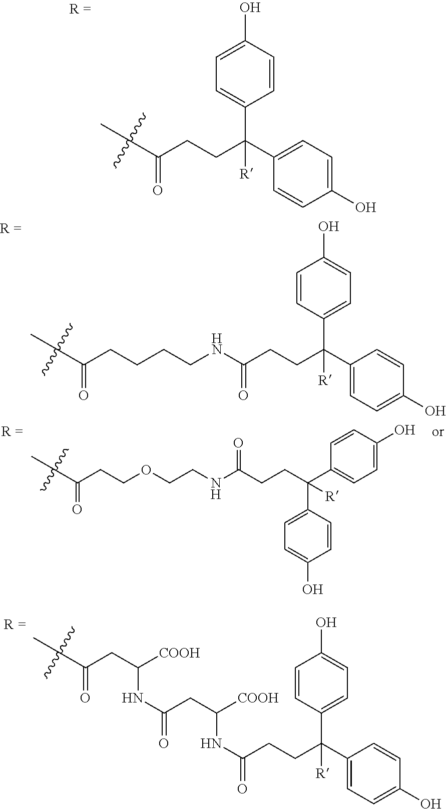

wherein R′ is H or C1-C7 alkyl, C1-C7 alkenyl, or C1-C7 heteroalkyl, optionally substituted with one, two or three substituents, and preferably R′ is methyl.

In another embodiment according to the invention, the “linker” (L) attaches the binding moiety (ligand B) to the drug moiety. The linker may be a bifunctional or a multifunctional moiety which can be used to link one or more drug moieties and binder moieties to form the SMDC. In embodiments, the conjugates of the present invention have a linker that links one drug moiety to one binding moiety (which may be univalent or multivalent).

Particularly preferred linkers are cleavable linkers. The most preferred cleavable linkers are dipeptides such as valine-citrulline and valine-alanine as disclosed in WO2015114171 and/or Cazzamalli et al., J Control Release (2017) 246: 39-45.

The drug moiety should stably remain attached to the ligand while in circulation but should be released when the conjugate reaches the site of disease.

Release mechanisms depend on a cleavable bond or other cleavable structure that is present in the linker. The cleavable structure may be similar to those specific to antibodies or other small molecules linked to cytotoxic payloads. Indeed the nature of the ligand is independent on that respect. Therefore we can envisage pH-dependent (Leamon, C P. et al. (2006) Bioconjugate Chem., 17, 1226; Casi, G. et al. (2012) J. Am. Chem. Soc, 134, 5887), reductive (Bernardes, G. J. et al. (2012) Angew. Chem. Int. Ed. Engl. 5 L 941; Yang, J. et al. (2006) Proc. Natl. Acad. Sci. USA, 103, 13872; Krall et al. (2014) Angew. Chem. Int. Ed., 53, 4231) and enzymatic release (Doronina S. O. et al. (2008) Bioconjugate Chem, 19, 1960; Sutherland, M. S. K. (2006) J. Biol. Chem, 281, 10540). In a specific setting, when functional groups are present on either the ligand or payloads (e.g., thiols, alcohols) which allow the creation of a cleavable bond, a linkerless connection can be established thus releasing intact payloads, which simplifies substantially pharmacokinetic analysis. A non-exhaustive list of moieties, which have cleavable bonds and which may be incorporated into linkers, is shown in the following table:

| | | Release |
|---|---|---|
| Linker | Structure | mechanism |
| Amide | 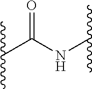 [SMILES: *NC(*)=O] 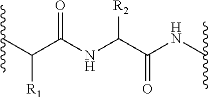 | Proteolysis |
| Ester | 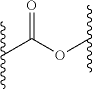 [SMILES: *OC(*)=O] | Hydrolysis |
| Carbamate | 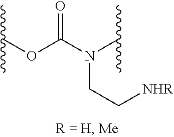 | Hydrolysis |
| Hydrazone | 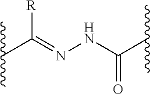 | Hydrolysis |
| Thiazolidine | 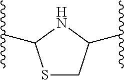 [SMILES: *C1CSC(*)N1] | Hydrolysis |
| Methylene alkoxy carbamate | 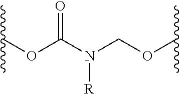 | Hydrolysis |
| Disulfide | 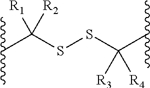 | Reduction |

wherein the substituents R and Rⁿ in the above formulas may suitably be independently selected from H, halogen, substituted or unsubstituted (hetero)alkyl, (hetero)alkenyl, (hetero)alkynyl, (hetero)aryl, (hetero)arylalkyl, (hetero)cycloalkyl, (hetero)cycloalkylaryl, heterocyclylalkyl, a peptide, an oligosaccharide or a steroid group. Suitably R and Rⁿ are independently selected from H, or C1-C7 alkyl or heteroalkyl. More suitably, R and Rⁿ are independently selected from H, methyl or ethyl.

Suitably, the conjugate is stable to hydrolysis. That is to say, less than about 10% of the conjugate undergoes hydrolysis in PBS pH 7.4 at 37° C. after 24 hours, as determined by HPLC. In these and other embodiments, the cleavable linker comprises a peptide unit that is specifically tailored so that it will be selectively enzymatically cleaved from the drug moiety by one or more proteases on the cell surface or the extracellular regions of the target tissue. The amino acid residue chain length of the peptide unit suitably ranges from that of a single amino acid to about eight amino acid residues. Numerous specific cleavable peptide sequences suitable for use in the present invention can be designed and optimized in their selectivity for enzymatic cleavage by a particular tumor-associated enzyme, e.g., a protease. Cleavable peptides for use in the present invention include those which are optimized toward the proteases MMP-1, 2 or 3, or cathepsin B, C or D. Especially suitable are peptides containing the sequence valine-citrulline (Val-Cit), which are cleavable by Cathepsin B. Cathepsin B is a ubiquitous cysteine protease. It is an intracellular enzyme, except in pathological conditions, such as metastatic tumors or rheumatoid arthritis. Therefore, non-internalizing SMDC of the present invention produced with cathepsin B-cleavable linkers are stable in circulation until activated in pathological tissue. Other suitable cleavable peptides are valine-alanine, valine-lysine or valine-arginine.

In one embodiment, the linker comprises a glucuronyl group that is cleavable by glucuronidase present on the cell surface or the extracellular region of the target tissue. It has been shown that lysosomal beta-glucuronidase is liberated extracellularly in high local concentrations in necrotic areas in human cancers, and that this provides a route to targeted chemotherapy (Bosslet, K. et al. Cancer Res. 58, 1195-1201 (1998)).

In these embodiments according to the invention, the linker moiety suitably further comprises, adjacent to the cleavable linker (e.g., a peptide sequence such as a dipeptide linkers), a “self-immolative” spacer (also called “suicide spacer”) portion.

The self-immolative spacers are also known as electronic cascade linkers. These linkers undergo elimination and fragmentation upon enzymatic cleavage of the peptide to release the drug in one of its active forms. The conjugate is stable extracellularly in the absence of an enzyme capable of cleaving the linker. However, upon exposure to a suitable enzyme, the linker is cleaved initiating a spontaneous self-immolative reaction resulting in the cleavage of the bond covalently linking the self-immolative moiety to the drug, to thereby effect release of the drug in one of its underivatized or pharmacologically active forms. In these embodiments, the self-immolative linker is coupled to the ligand moiety through an enzymatically cleavable peptide sequence that provides a substrate for an enzyme to cleave the amide bond to initiate the self-immolative reaction. Suitably, the drug moiety is connected to the self-immolative moiety of the linker via a chemically reactive functional group naturally present in the drug structure or in an active derivative thereof, such as a primary or secondary amine, hydroxyl, sulfhydryl or carboxyl group.

Examples of self-immolative linkers are PABC or PAB (para-aminobenzyloxycarbonyl), attaching the drug moiety to the ligand in the conjugate (Carl et al. (1981) J. Med. Chem. 24: 479-480; Chakravarty et al. (1983) J. Med. Chem. 26: 638-644). The amide bond linking the carboxy terminus of a peptide unit and the para-aminobenzyl of PAB may be a substrate and cleavable by certain proteases. The aromatic amine becomes electron-donating and initiates an electronic cascade that leads to the expulsion of the leaving group, which releases the free drug after elimination of carbon dioxide (de Groot et al. (2001) Journal of Organic Chemistry 66 (26): 8815-8830). Further examples of self-immolating linkers are described in WO2005/082023. Suitably, the linker may be polar or charged in order to improve water solubility of the conjugate. For example, the linker according to the invention may further comprise a “peptidic spacer” (S) from about 1 to about 20, suitably from about 2 to about 10, residues of one or more known water-soluble oligomers such as peptides, oligosaccharides, glycosaminoglycans, polyacrylic acid or salts thereof, polyethylene glycol, polyhydroxyethyl (meth) acrylates, polysulfonates, etc. Suitably, the linker may comprise a polar or charged peptide moiety comprising, e.g., from 2 to 10 amino acid residues Amino acids may refer to any natural or non-natural amino acid. The peptide linker suitably includes a free thiol group, preferably a C-terminal cysteine, for forming the said cleavable disulfide linkage with a thiol group on the drug moiety. A suitable peptidic spacer of this type is -Asp-Arg-Asp-Cys-(SEQ ID NO: 26). In these embodiments according to the invention, the linker suitably further may comprise a spacer unit linked for example, to a peptidic spacer and/or cleavable linker, for example via an amide, amine or thioether bond. The spacer unit is of a length that enables, e.g., the cleavable peptide sequence to be engaged by the cleaving enzyme (e.g., cathepsin B) and suitably also the hydrolysis of the amide bond coupling the cleavable peptide to the self-immolative moiety X. Spacer units may for example comprise a divalent radical such as alkylene, arylene, a heteroarylene, repeating units of alkyloxy (e.g., polyethylenoxy, PEG, polymethyleneoxy) and alkylamino (e.g., polyethyleneamino), or diacid ester and amides including succinate, succinamide, diglycolate, malonate, and caproamide. A preferred spacer unit is an alkyl- or polyethyleneglycol-based linking moiety. A linker of the invention may comprise, for example,—a peptidic spacer—a spacer unit—cleavable linker—“self-immolative” spacer.

Such linker, wherein the peptidic spacer is linked to the C terminal of the binding moiety and the “self-immolative” spacer is linked to the drug. An example of this linker may be found in the compound 2 of the invention.

Alternatively, the linker may be a chelator suitable for indirect radiolabeling with, e.g., indium, yttrium, lanthanides or technetium and rhenium. The chelating ligand, preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis (2-aminoethyl)-N,N′,N″,N′″-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N, N′, N″-triacetic acid (NOTA), 1,4,8,11-tetraazacyclotetradecane-N, N′, N″, N′″-tetraacetic acid (TETA), mercaptoacetyl diglycine (MAG₂), mercaptoacetyl triglycine (MAG₃), mercaptoacetyl glycyl cysteine (MAGC), cysteinyl glycyl cysteine (CGC) to either amine or thiol groups of the specific binding moiety. The chelating ligands possess a suitable coupling group, e.g., active esters, maleimides, thiocarbamates or α-halogenated acetamide moieties.

In one embodiment according to the invention, the “drug moiety” (D) may be a therapeutic agent such as a biocidal molecule, a cytotoxic agent, chemotherapeutic agent, an anti-hormonal agent, a radioisotope, a photosensitizer, an enzyme, a hormone, or a DNA-damaging agent. In one embodiment according to the invention, the “drug moiety” (D) is a cytotoxic agent that inhibits or prevents the function of cells and/or causes destruction of cells. Examples of cytotoxic agents include chemotherapeutic agents, and toxins such as small molecule toxins or enzymatically active toxins of bacterial, fungal, plant or animal origin, including synthetic analogues and derivatives thereof. The cytotoxic agent may be selected from the group consisting of an auristatin, a DNA minor groove binding agent, a DNA minor groove alkylating agent, a tubulin disruptor, an enediyne, a lexitropsin, a duocarmycin, a taxane, anthracyclines, a puromycin, a dolastatin, a maytansinoid and a vinca alkaloid or a combination of two or more thereof.

In one embodiment according to the invention, the drug is a chemotherapeutic agent selected from the group consisting of a topoisomerase inhibitor; an alkylating agent (e.g., nitrogen mustards); ethylenimes; alkylsulfonates; triazenes; piperazines; and nitrosureas), an antimetabolite (e.g., mercaptopurine, thioguanine, 5-fluorouracil); an antibiotic (e.g., anthracyclines, dactinomycin, bleomycin, adriamycin, mithramycin. dactinomycin); a mitotic disrupter (e.g., plant alkaloids such as vincristine and/or microtubule antagonists such as paclitaxel); a DNA intercalating agent (e.g., carboplatin and/or cisplatin); a DNA synthesis inhibitor; a DNA-R A transcription regulator; an enzyme inhibitor; a gene regulator; a hormone response modifier; a hypoxia-selective cytotoxin (e.g., tirapazamine); an epidermal growth factor inhibitor; an anti-vascular agent (e.g., xanthenone 5,6-dimethylxanthenone-4-acetic acid); a radiation-activated prodrug (e.g., nitroarylmethyl quaternary (NMQ) salts); or a bioreductive drug or a combination of two or more thereof.

The chemotherapeutic agent may selected from the group consisting of Erlotinib (TARCEVA®), Bortezomib (VELCADE®), Fulvestrant (FASLODEX®), Sutent (SU1 1248), Letrozole (FEMARA®), Imatinib mesylate (GLEEVEC®), PTK787/ZK 222584, Oxaliplatin (Eloxatin®), 5-FU (5-fluorouracil), Leucovorin, Rapamycin (Sirolimus, RAPAMUNE®), Lapatinib (GSK572016), Lonafarnib (SCH 66336), Sorafenib (BAY43-9006), and Gefitinib (IRESSA®), AG1478, AG1571 (SU 5271; Sugen) or a combination of two or more thereof.

The chemotherapeutic agent may be an alkylating agent such as thiotepa, CYTOXAN® and/or cyclosphosphamide; an alkyl sulfonate such as busulfan, improsulfan and/or piposulfan; an aziridine such as benzodopa, carboquone, meturedopa and/or uredopa; ethylenimines and/or methylamelamines such as altretamine, triethylenemelamine, triethylenepbosphoramide, triethylenethiophosphoramide and/or trimethylomelamine; acetogenin such as bullatacin and/or bullatacinone; camptothecin; bryostatin; callystatin; cryptophycins; dolastatin; duocarmycin; eleutherobin; pancratistatin; sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide and/or uracil mustard; nitrosureas such as carmustine, chlorozotocin, fotemustine, lomustine, nimustine, and/or ranimnustine; dynemicin; bisphosphonates such as clodronate; an esperamicin; a neocarzinostatin chromophore; aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN®. doxorubicin such as morpholino-doxorubicin, cyanomorpholino-doxorubicin, 2-pyrrolino-doxorubicin and/or deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; macrocyclic depsipeptides such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofiran; spirogermanium; tenuazonic acid; triaziquone; 2,2′,2″-trichlorotriethylamine; trichothecenes such as verracurin A, roridin A and/or anguidine; urethan; vindesine; dacarbazine; mannomustine; mitobronitol; mitolactol; pipobroman; gacytosine; arabinoside; cyclophosphamide; thiotepa; taxoids such as TAXOL®. paclitaxel, abraxane, and/or TAXOTERE®, doxetaxel; chloranbucil; GEMZAR®. gemcitabine; 6-thioguanine; mercaptopurine; methotrexate; platinum analogues such as cisplatin and carboplatin; vinblastine; platinum; etoposide; ifosfamide; mitoxantrone; vincristine; NAVELBINE®, vinorelbine; novantrone; teniposide; edatrexate; daunomycin; aminopterin; xeloda; ibandronate; topoisomerase inhibitor RFS 2000; difluoromethylomithine (DMFO); retinoids such as retinoic acid; capecitabine; and pharmaceutically acceptable salts, acids, derivatives or combinations of two or more of any of the above.

The drug may be a tubulin disruptor including but are not limited to: taxanes such as paclitaxel and docetaxel, vinca alkaloids, discodermolide, epothilones A and B, desoxyepothilone, cryptophycins, curacin A, combretastatin A-4-phosphate, BMS 247550, BMS 184476, BMS 188791; LEP, RPR 109881A, EPO 906, TXD 258, ZD 6126, vinflunine, LU 103793, dolastatin 10, monomethyl auristatin E (MMAE), monomethyl auristatin F (MMAF), E7010, T138067 and T900607, colchicine, phenstatin, chalcones, indanocine, T 138067, oncocidin, vincristine, vinblastine, vinorelbine, vinflunine, halichondrin B, isohomohalichondrin B, ER-86526, pironetin, spongistatin 1, spiket P, cryptophycin 1, LU103793 (cematodin or cemadotin), rhizoxin, sarcodictyin, eleutherobin, laulilamide, VP-16 and D-24851 and pharmaceutically acceptable salts, acids, derivatives or combinations of two or more of any of the above.

The drug may be a DNA intercalator including but are not limited to: acridines, actinomycins, anthracyclines, benzothiopyranoindazoles, pixantrone, crisnatol, brostallicin, CI-958, doxorubicin (adriamycin), actinomycin D, daunorubicin (daunomycin), bleomycin, idarubicin, mitoxantrone, cyclophosphamide, melphalan, mitomycin C, bizelesin, etoposide, mitoxantrone, SN-38, carboplatin, cis-platin, actinomycin D, amsacrine, DACA, pyrazoloacridine, irinotecan and topotecan and pharmaceutically acceptable salts, acids, derivatives or combinations of two or more of any of the above.

The drug may be an anti-hormonal agent that acts to regulate or inhibit hormone action on tumors such as anti-estrogens and selective estrogen receptor modulators, including, but not limited to, tamoxifen, raloxifene, droloxifene, 4-hydroxytamoxifen, trioxifene, keoxifene, LY1 17018, onapristone, and/or fareston toremifene and pharmaceutically acceptable salts, acids, derivatives or combinations of two or more of any of the above. The drug may be an aromatase inhibitor that inhibits the enzyme aromatase, which regulates estrogen production in the adrenal glands such as, for example, 4(5)-imidazoles, aminoglutethimide, megestrol acetate, AROMASIN®. exemestane, formestanie, fadrozole, RIVISOR®. vorozole, FEMARA®. letrozole, and ARIMIDEX® and/or anastrozole and pharmaceutically acceptable salts, acids, derivatives or combinations of two or more of any of the above.

The drug may be an anti-androgen such as flutamide, nilutamide, bicalutamide, leuprolide, goserelin and/or troxacitabine and pharmaceutically acceptable salts, acids, derivatives or combinations of two or more of any of the above.

The drug may be a protein kinase inhibitor, a lipid kinase inhibitor or an anti-angiogenic agent.

In a preferred embodiment, the drug is a maytansinoid, in particular DM1, or a tubulin disruptor. Preferably, the drug in its active form comprises a thiol group, whereby a cleavable disulfide bond may be formed through the sulfur of the thiol group to bond the drug to the linker moiety in the conjugates of the invention.

In a preferred embodiment, the drug is monomethyl auristatin E (MMAE).

In addition to the drug moieties disclosed WO2015114171, other cytotoxic agents coupled to the targeting moiety of the SMDC are useful in the present invention, including in another embodiment PNU-159682 (a nemorubicin metabolite) (disclosed in Cazzamalli et al., Mol Cancer Ther., (2016) 15(12): 2926-2935) or MMAF.

In one embodiment the drug moiety (D) can be a radionuclide such as radioisotopes of Technetium, Indium, Yttrium, Copper, Lutetium or Rhenium in particular, ⁹⁴mTc, ⁹⁹mTc, ¹⁸⁶Re, ¹⁸⁸Re, ¹¹¹In, ⁸⁶Y, ⁸⁸Y, ¹⁷⁷Lu, ²²³Ra, ⁸⁹Sr, ²⁰³Pb, ⁴⁷Sc, 64Cu and ⁶⁷Cu, which may be attached to binding moiety of the invention using conventional chemistry known in the art of antibody imaging. The specific binding moiety disclosed herein are particularly well suited for radiolabeling with isotopes such as ^{94m}Tc, ^{99m}Tc, ¹⁸⁶Re, ¹⁸⁸Re, ²⁰³Pb, ⁶⁷Ga, ⁶⁸Ga, ⁴³Sc, ⁴⁷Sc, ¹¹⁰mIn, ¹¹¹In, ⁹⁷Ru, ⁶²Cu, ⁶⁴Cu, ⁶⁷Cu, ⁶⁸Cu, ⁸⁶Y, ⁸⁸Y, ⁹⁰Y, ¹²¹Sm, ¹⁶¹Tb, ¹⁵³Sm, ¹⁶⁶Ho, ¹⁰⁵Rh, ¹⁷⁷Lu, ⁷²Lu, ¹⁸F, ²²³Ra, ⁸⁹Sr, ¹⁵³Sm, ¹²³I, ¹²⁴I, ¹²⁵I, ¹³¹I, ²¹¹At and ²²⁵Ac and subsequent use in radio-diagnosis and radiotherapy. Preferably, beta-emitters, such as ⁸⁹Sr, ⁹⁹Y, ¹³¹I, and ¹⁷⁷Lu, are used for therapeutic applications. Alpha-emitters, such as ²¹¹At, ²²⁵Ac and ²²³Ra may also be used for therapy.

In one embodiment according to the invention, the “drug moiety” (D) may be an imaging, detectable, diagnostic agent such as a fluorescent label or a radioisotope, e.g., a non-therapeutic radioisotope.

In one embodiment the radioisotope is ⁹⁹mTc.

In one embodiment the fluorescent label is IRDye 800CW (Licor).

The drug may be used in unmodified or modified form. Combinations of drugs in which some are unmodified and some are modified may be used. For example, the drug may be chemically modified. One form of chemical modification is the derivatisation of a carbonyl group—such as an aldehyde. In one embodiment, the drug is modified to allow the incorporation of the linker. For example, a drug comprising a hydroxyl group may be converted to the corresponding 2-ethanethiol carbonate or 2-ethanethiol carbamate thereby introducing thiol groups for disulphide linkage.

In one embodiment, the SMDC comprises a compound having the following structure:

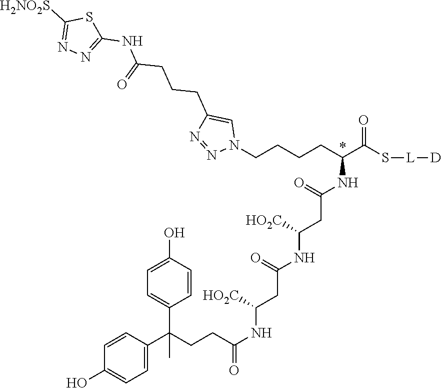

wherein S, L and D are defined as above. The linker may also comprise a spacer unit as defined above.

When there is an amine substituent of the a (alpha) carbon indicated with *, it is preferred that the chiral amine in the (S) configuration, although it may also be in the (R) configuration, e.g.:

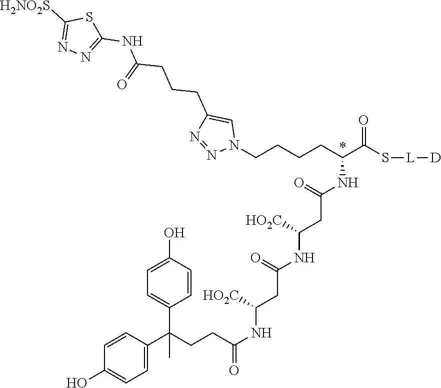

In another embodiment, the SMDC comprises a compound having the following structure:

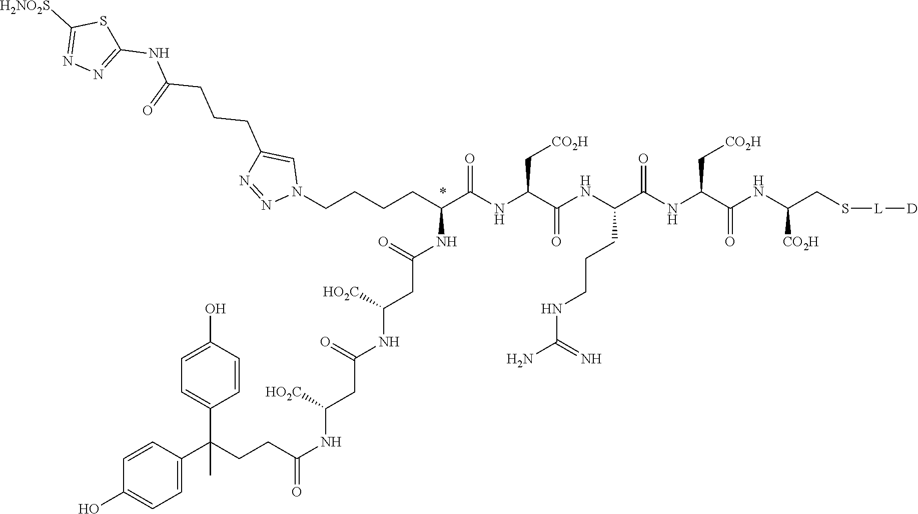

wherein L and D are defined as above. The linker may also comprise a spacer unit as defined above.

When there is an amine substituent of the a (alpha) carbon indicated with *, it is preferred that the chiral amine in the (S) configuration, although it may also be in the (R) configuration, e.g.:

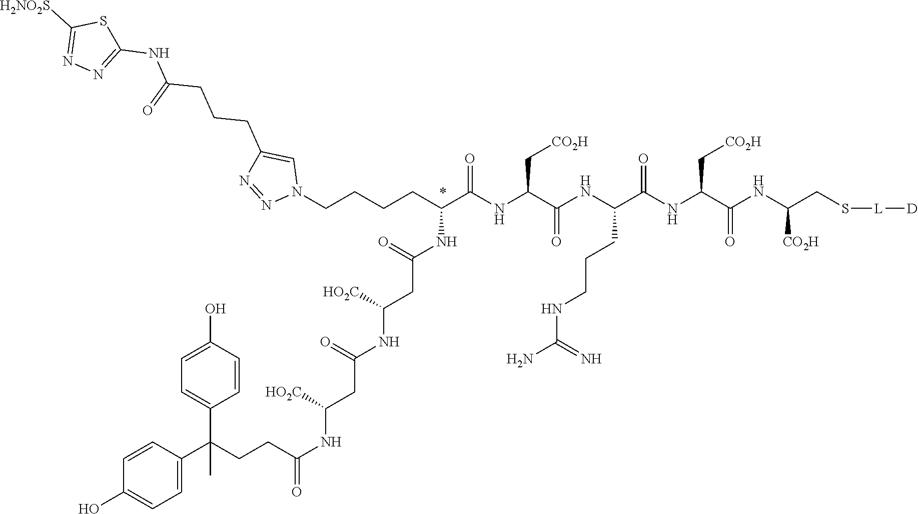

wherein L is a linker comprising a cleavable linker, e.g., a dipeptide cleavable linker such as valine-citrulline, or valine-alanine, and/or a self-immolative spacer (or self-immolative linker), e.g., para-aminobenzyloxycarbonyl (PABC); the linker may also comprise a spacer unit, e.g., an alkyl- or polyethyleneglycol-based linking moiety; and wherein D is cytotoxic drug, e.g., a tubulin inhibitor or a DNA-damaging agent, such as MMAE, MMAF, or PNU-159682. Suitable self-immolative linker are known in the art, e.g., as disclosed in WO2015114171, and Bouchard et al., Bioorganic & Medicinal Chemistry Letters 24 (2014) 5357-5363.

In another embodiment the SMDC is comprises a compound having the following structure:

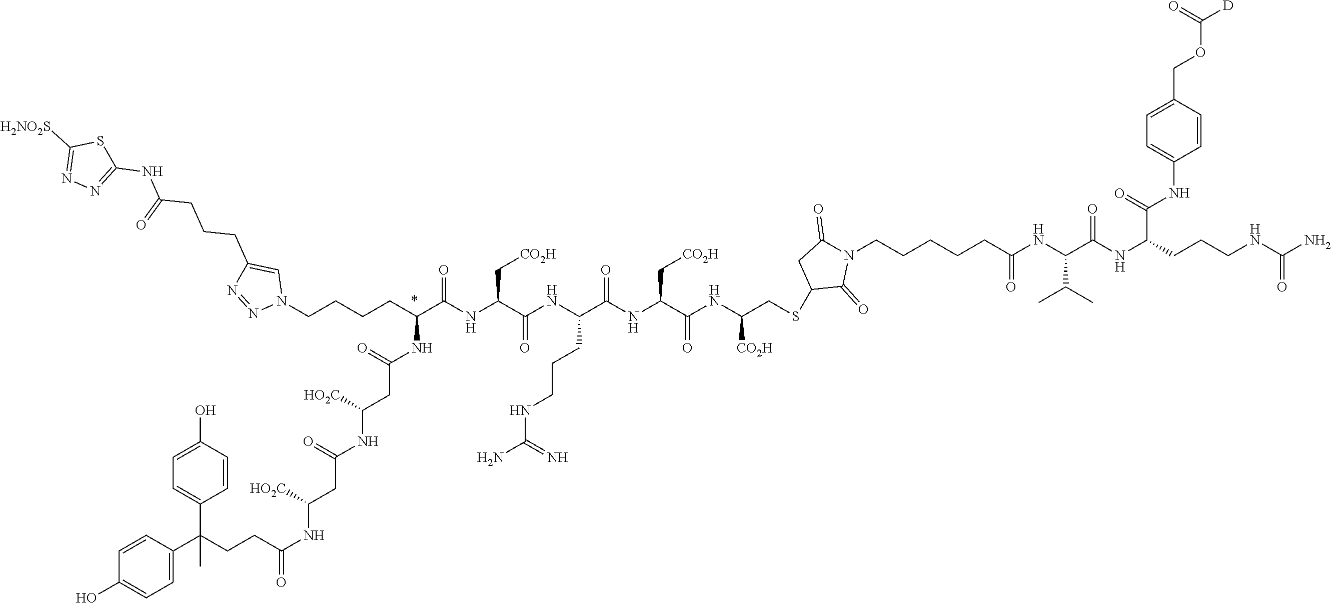

wherein D is defined as above.

When there is an amine substituent of the α(alpha) carbon indicated with *, it is preferred that the chiral amine in the (S) configuration, although it may also be in the (R) configuration, e.g.:

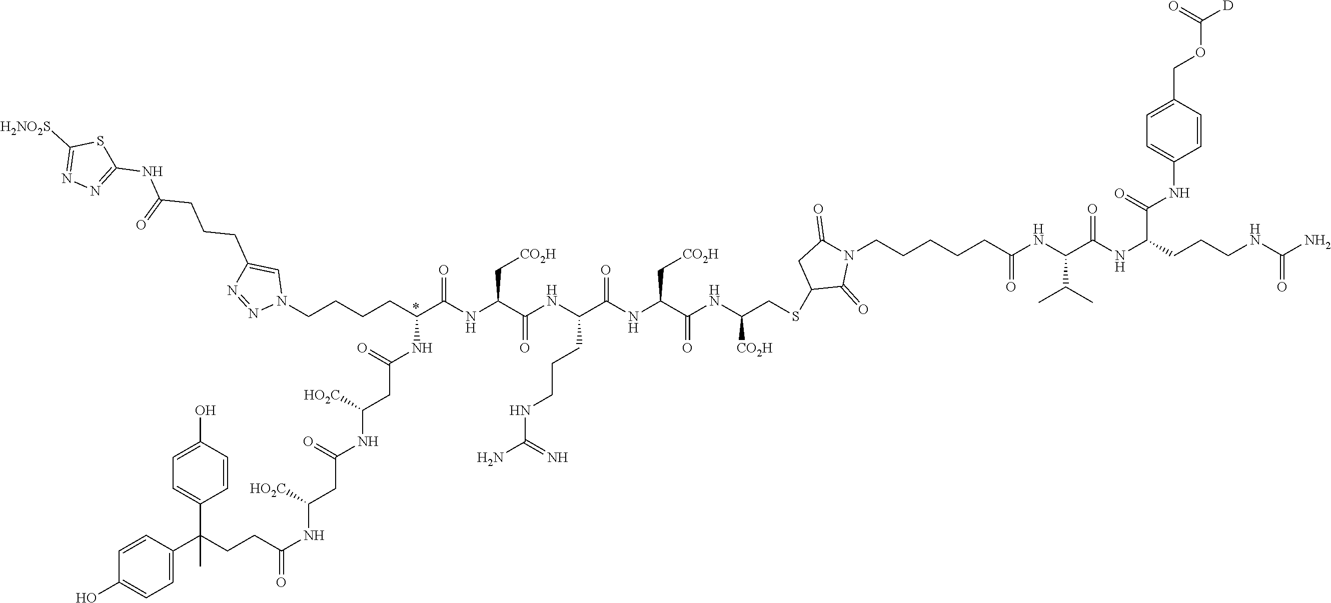

wherein D is defined as above.

In another preferred embodiment the SMDC comprises a compound having the following structure:

Compound 2:

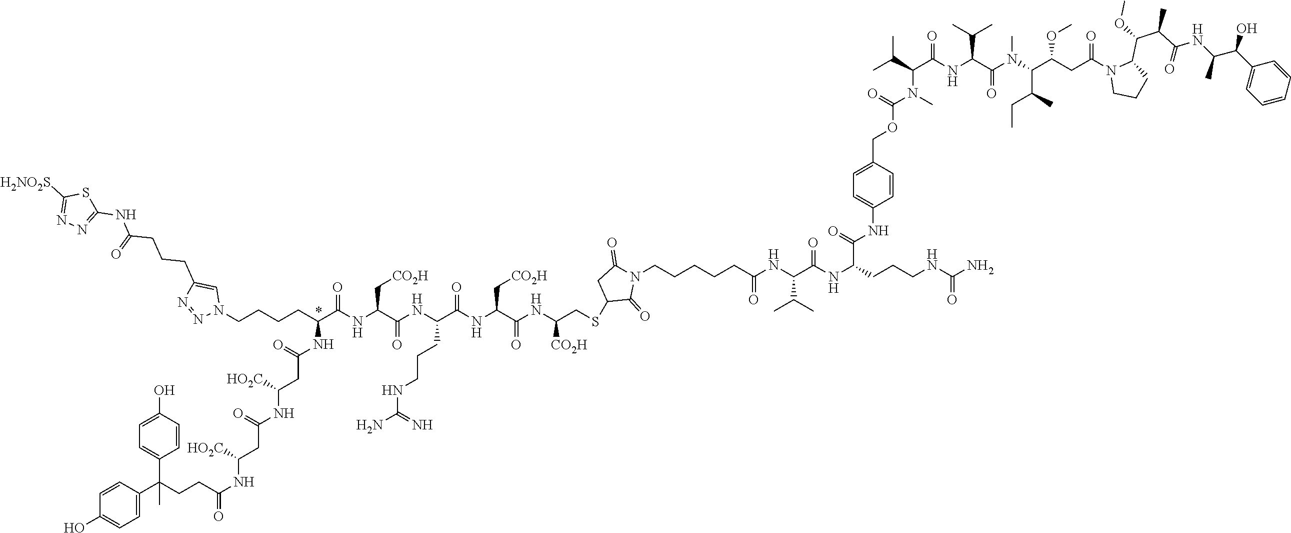 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC[C@H](NC(=O)[C@H](CC(=O)O)NC(=O)[C@H](CCCNC(=N)N)NC(=O)[C@H](CC(=O)O)NC(=O)[C@H](CCCCn3cc(CCCC(=O)Nc4nnc(S(N)(=O)=O)s4)nn3)NC(=O)C[C@H](NC(=O)C[C@H](NC(=O)CCC(C)(c3ccc(O)cc3)c3ccc(O)cc3)C(=O)O)C(=O)O)C(=O)O)C2=O)C(C)C)cc1)C(C)C]

When there is an amine substituent of the a (alpha) carbon indicated with *, it is preferred that the chiral amine in the (S) configuration, although it may also be in the (R) configuration, e.g.:

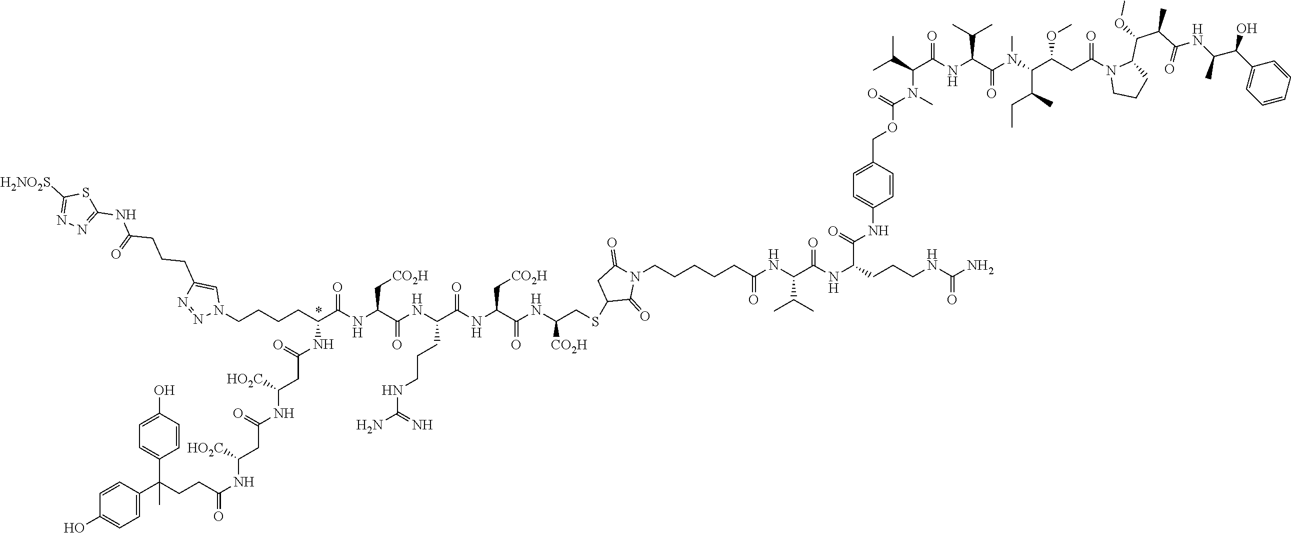 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC[C@H](NC(=O)[C@H](CC(=O)O)NC(=O)[C@H](CCCNC(=N)N)NC(=O)[C@H](CC(=O)O)NC(=O)[C@@H](CCCCn3cc(CCCC(=O)Nc4nnc(S(N)(=O)=O)s4)nn3)NC(=O)C[C@H](NC(=O)C[C@H](NC(=O)CCC(C)(c3ccc(O)cc3)c3ccc(O)cc3)C(=O)O)C(=O)O)C(=O)O)C2=O)C(C)C)cc1)C(C)C]

In another aspect, the present invention provides in addition to the composition comprising an immunocytokine and a targeted therapeutic agent, a treatment for a neoplastic disease, preferably for the treatment of a neoplastic disease, including a tumor, in particular a solid tumor, in particular for the treatment of renal cell carcinoma or a colorectal cancer, comprising administering the composition of the invention.

In another aspect, the invention provides a composition comprising an immunocytokine and a targeted therapeutic agent for use in the treatment of neoplastic diseases including a tumor, in particular a solid tumor or CAIX expressing tumor, in particular for the treatment of renal cell carcinoma or a colorectal cancer.

In another aspect, the invention provides use of a composition comprising

(i) an immunocytokine and

(ii) a small molecule drug conjugate (SMDC),

wherein

the immunocytokine comprises an antibody or antibody fragment conjugated to a cytokine, and

the small molecule comprises a moiety capable of binding to a tumor-associated target, in the manufacture of a medicament for the treatment of a neoplastic disease.

“Neoplastic disease” in accordance with the invention is a disease or disorder such as cancer that can be treated via the targeted delivery of cytotoxic agents. Non-limiting examples of cancers that may be treated include benign and malignant tumors; leukemia and lymphoid malignancies, including breast, ovarian, stomach, endometrial, salivary gland, lung, kidney, colon, thyroid, pancreatic, prostate or bladder cancer. The disease may be a neuronal, glial, astrocytal, hypothalamic or other glandular, macrophagal, epithelial, stromal and blastocoelic disease; or inflammatory, angiogenic or an immunologic disease. An exemplary disease is a solid, malignant tumor.

The terms “cancer,” “cancerous,” and “tumor” are used in their broadest sense as generally meaning the physiological condition in mammals that is typically characterized by unregulated cell growth. A tumor comprises one or more cancerous cells. Examples of cancer include, but are not limited to, carcinoma, lymphoma, blastoma, sarcoma, and leukemia or lymphoid malignancies. Further examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, gastrointestinal stromal tumor (GIST), pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, as well as head and neck cancer. Based on established evidence of expression of CAIX, it is expected that the present invention will be suitable in particular for the treatment of glioblastoma, lung cancer, head and neck cancer, cervical cancer, breast cancer, and, especially, renal cell carcinoma and colorectal cancer.

In another aspect, the present invention provides a pharmaceutical composition comprising an immunocytokine and a targeted therapeutic agent according to the first aspect of the invention.

In another aspect, the present invention provides a product, for use in combination with an immunocytokine, comprising a compound of Formula (I) as defined herein and a cleavage agent for cleaving said cleavable linker L, as a combined preparation for sequential administration in the treatment of cancer.

In another aspect, the present invention provides a method of treating a neoplastic disease, preferably a solid tumor or a CAIX-expressing tumor such as renal cell carcinoma, comprising administering an effective amount of a combination of pharmaceutical compositions comprising an immunocytokine and a SMDC to a patient in need thereof. In particular embodiments, the administration of said SMDC pharmaceutical composition is followed after a suitable interval of time by administration of a cleavage agent for cleaving said cleavable linker L.

In another aspect, the present invention provides a composition comprising an immunocytokine and a targeted therapeutic agent for use in a method of treatment of a neoplastic disease, preferably a solid tumor or a CAIX-expressing tumor such as renal cell carcinoma or colorectal cancer.

In another aspect, the present invention also provides the use of a composition comprising an immunocytokine and a targeted therapeutic agent for the manufacture of a medicament for the treatment of a neoplastic disease, preferably a solid tumor or a CAIX-expressing tumor such as renal cell carcinoma or colorectal cancer.

It is generally convenient to provide the immunocytokine and the SMDC as separate molecules. They may be provided as a combined preparation, or as separate formulations to permit either simultaneous or sequential administration. The clinician can determine the most suitable manner of administering the each dose of the immunocytokine and the SMDC to the patient. For example, the method of treatment may comprise injecting the immunocytokine and SMDC in separate injections, simultaneously or sequentially. Where sequential administration is used, the immunocytokine and SMDC are preferably injected within 24 hours, 12 hours, 1 hour or more preferably within 30 minutes of each other. In one embodiment, the immunocytokine is administered first; in another embodiment, the SMDC is administered first. The immunocytokine and SMDC may be injected at the same place, e.g., at the point in tumor site, or at different points. A combined injection of the immunocytokine and SMDC may be administered. It may be preferable to administer a dose in multiple injections, for example to inject multiple locations across a tumor or around a tumor site, or to facilitate administration of a larger volume of immunocytokine and/or SMDC.

The dose is an amount of immunocytokine and/or SMDC, administered at one time, effective to treat a tumor in the combination therapy according to the invention. A single dose may be administered in a treatment period of 1 hour or less, preferably in a period of 30 minutes or less, e.g. 15, 10, 5 or 1 minute or less.

The quantity of immunocytokine and/or SMDC administered will depend on the type and severity of the disease, e.g., the size and nature of the tumor, among other factors. For example, the dose of IL2-scFv immunocytokine may be in the range of 10-100 μg, e.g., 20-40 μg. Corresponding doses using other immunocytokine formats may be straightforwardly calculated to administer an appropriate quantity of cytokine. These are examples only and, of course, different doses may be used.

Similarly, depending on the type and severity of the disease, between about 1 μg/kg to 15 mg/kg of SMDC may be used as an initial candidate dosage for administration to the patient, whether, for example, by one or more separate administrations, or by continuous infusion. A typical daily dosage might range from about 1 μg/kg to 100 mg/kg or more. An exemplary dosage of SMDC may be in the range of about 0.1 to about 10 mg/kg of patient weight.

The clinician will determine a therapeutically effective amount of each component of the therapeutic composition for administration.

It was observed that, surprisingly, that in addition to the complete eradication of tumors provided by the method of the present invention, the tumor eradication was persistent. Thus, contrary to the vast majority of methods of treating tumors, no relapse was observed. Thus, in another embodiment, the invention provides a vaccine which can be administered to patients in need thereof, either prophylactically or after treatment of actual disease, which prevents the development or relapse of neoplastic disease, e.g., in which IL2 and/or CAIX are overexpressed.

In another aspect, the present invention provides a method of imaging, detecting, or diagnosing a disease or disorder in a patient. A method of imaging, detecting, or diagnosing a disease or disorder comprising administering a SMDC as described herein to a patient is similarly contemplated. The disease or disorder may be chronic and acute disorders or diseases including those pathological conditions which predispose to the disorder. One particular disease that is applicable to imaging, detecting or diagnosing by the present invention is a solid tumor or a CAIX-expressing tumor such as kidney cancer or colorectal cancer.

### Examples

### Example 1: Preparation of CAIX-Binder-Based Small Molecule Drug Conjugate. General Remarks and Procedures

Peptide grade N, N-dimethylformamide (DMF) for solid phase synthesis was bought from ABCR. All other solvents were used as supplied by Fisher Chemicals, Merck or Sigma Aldrich in HPLC or analytical grade. H-Cys(Trt)-2-CT-polystyrene resin was purchased from RAPP Polymere. Maleimidocaproyl-ValCit-p-aminobenzylalcohol-MMAE was purchased from Levena Biopharma (No.9 Weidi Road, Qixia District, Nanjing, 210046, China). L19IL2 was produced by Philogen S.p.A. (Siena, Italy) and diluted to the concentration used for therapy studies with the appropriate formulation buffer (Philogen). All other reagents were purchased from Sigma Aldrich, Acros, ABCR or TCI and used as supplied.

Yields refer to chromatographically purified compounds. High-Resolution Mass Spectrometry (HRMS) spectra and analytical Reversed-Phase Ultra Performance Liquid Chromatography (UPLC) were recorded on a Waters Xevo G2-XS QTOF coupled to a Waters Acquity UPLC H-Class System with PDA UV detector, using a ACQUITY UPLC BEH C18 Column, 130 Å, 1.7 μm, 2.1 mm×50 mm at a flow rate of 0.6 ml min′ with linear gradients of solvents A and B (A=Millipore water with 0.1% formic acid [FA], B=MeCN with 0.1% formic acid [FA]). Preparative reversed-phase high-pressure liquid chromatography (RP-HPLC) were performed on a Waters Alliance HT RP-HPLC with PDA UV detector, using a Synergi 4 μm, Polar-RP 80 Å 10×150 mm C18 column at a flow rate of 4 ml min⁻¹ with linear gradients of solvents A and B (A=Millipore water with 0.1% trifluoroacetic acid [TFA], B=MeCN with 0.1% trifluoroacetic acid [TFA]).

### Synthesis of CAIX Targeted SMDC

### Synthesis of AAZ*-SMDC Precursor (Compound 1) in S Configuration

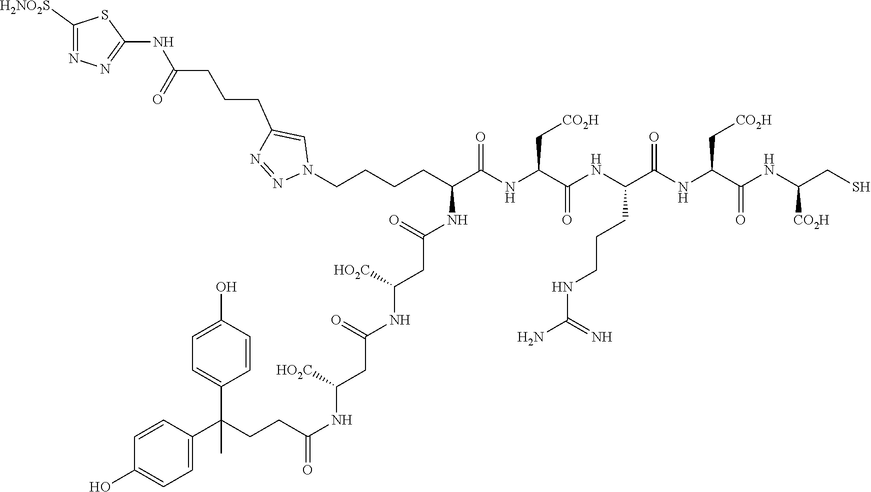 [SMILES: CC(CCC(=O)N[C@@H](CC(=O)N[C@@H](CC(=O)N[C@@H](CCCCn1cc(CCCC(=O)Nc2nnc(S(N)(=O)=O)s2)nn1)C(=O)N[C@@H](CC(=O)O)C(=O)N[C@@H](CCCNC(=N)N)C(=O)N[C@@H](CC(=O)O)C(=O)N[C@@H](CS)C(=O)O)C(=O)O)C(=O)O)(c1ccc(O)cc1)c1ccc(O)cc1]

Chemical Formula: C₅₆H₇₅N₁₇O₂₂S₃

### Molecular Weight: 1434.4

Commercially available pre-loaded Fmoc-Cys(Trt)-OH on polystyrene resin (300 mg, 0.19 mmol) was swollen in DMF for 15 min. The Fmoc group was removed with 20% piperidine in DMF (3×10 min×10 ml) and the resin washed with DMF (4×10 min×10 ml). Fmoc-Asp(OtBu)-OH (233 mg, 0.57 mmol, 3 eq) was activated with HATU (215 mg, 0.57 mmol, 3 eq), and DIPEA (197 μl, 1.13 mmol, 6 eq) in DMF (3 ml) at 0° C. for 15 min and then reacted with the resin for 1 h under gentle agitation. After washing the resin with DMF (4×10 min×10 ml) the Fmoc group was removed with 20% piperidine in DMF (3×10 min×10 ml) and the resin washed with DMF (4×10 min×10 ml) before the peptide was extended with Fmoc-Arg(Pbf)-OH (368 mg, 0.57 mmol, 3 eq), Fmoc-Asp(OtBu)-OH (233 mg, 0.57 mmol, 3 eq), Fmoc-Lys(N3)-OH (224 mg, 0.57 mmol, 3 eq), Fmoc-Asp-OtBu (233, 0.57 mmol, 3 eq), Fmoc-Asp-OtBu (233, 0.57 mmol, 3 eq) and 4,4-bis(4-hydroxyphenyl)valeric acid (162 mg, 0.57 eq, 3 eq) in the indicated order using the same coupling conditions (HATU/DIPEA), Fmoc-deprotection (20% piperidine in DMF) and washing step with DMF mentioned before.

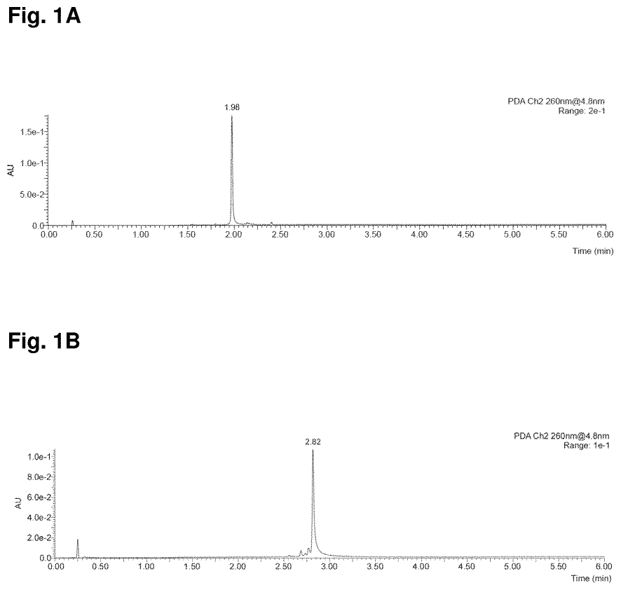

After the last coupling step, a solution of CuI (11 mg, 0.06 mmol, 0.3 eq), TBTA (10 mg, 0.02 mmol, 0.1 eq) and N-(5-sulfamoyl-1,3,4-thiadiazol-2-yl)hex-5-ynamide (155 mg, 0.57 mmol, 3 eq) in a mixture of DMF (1.5 ml) and THF (1.5 ml) was prepared and reacted with the resin at room temperature for 48 h. After washing with DMF (4×10 min×10 ml), EDTA 50 mM (4×10 mM×10 ml) and DCM (4×10 mM×10 ml), the compound was cleaved from the resin by agitating with a mixture of TFA (6 ml), TIS (1.1 ml), H₂O (300 μl), m-Cresol (300 μl) and Thioanisol (300 μl) at room temperature for 1 h. Cleavage solution was added drop-wise to ice cold diethyl ether (50 ml) obtaining a white precipitate. The pellet was collected by centrifugation, dried under vacuum, redissolved in in a 1:1 H₂O/MeCN mixture (1 ml), and added with an excess of Tris(2-carboxyethyl)phosphine hydrochloride (30 eq). The product was purified by reversed-phase HPLC (Synergi RP Polar, 5% MeCN in 0.1% aq. TFA to 80% over 20 min). After lyophilization the final compound was collected as a white powder (25 mg, 17.4 μmol, 9% yield). See FIG. 1A, which demonstrates analytical UPLC trace of compound 1 on a BEH C18 Column, 130 Å, 1.7 μm, 2.1 mm×50 mm at a flow rate of 0.6 ml min⁻¹, 5% MeCN in 0.1% aq. FA to 80% MeCN in 6 min.

MS (ESI) m/z calcd. for [C₁₂₄H₁₈₂N₂₈O₃₇S₃]¹⁺: 1434.4435 [M+H]¹⁺, found: 1434.4501; m/z calcd. for [C₁₂₄H₁₈₃N₂₈O₃₇S₃]²⁺: 717.7218 [M+2H]²⁺, found: 717.7317.

### Synthesis of AAZ*-ValCit-MMAE (Compound 2) in S Configuration

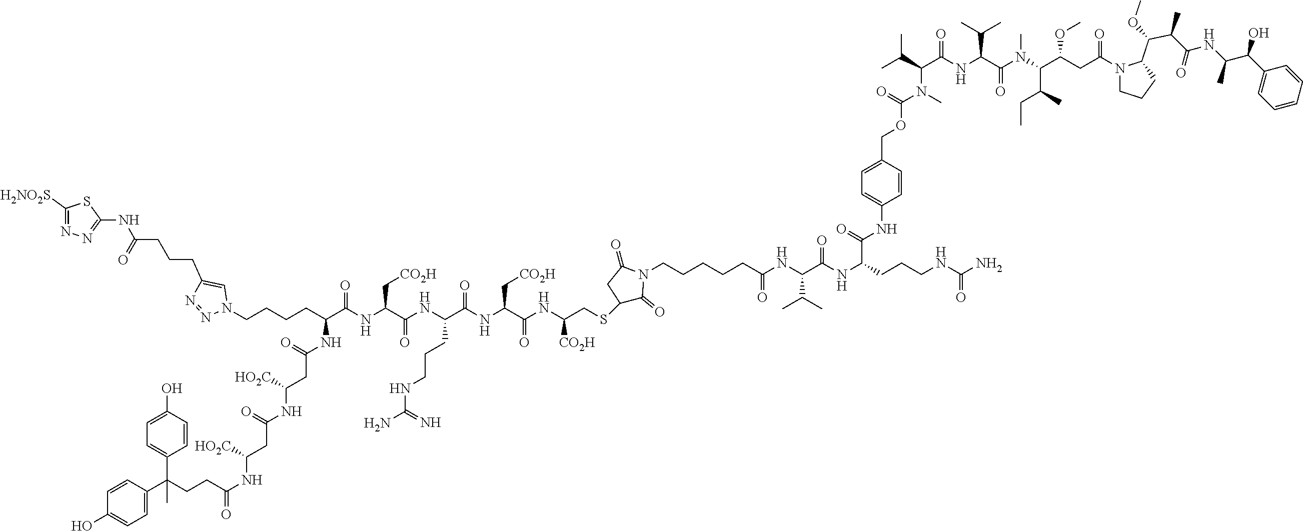 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC[C@H](NC(=O)[C@H](CC(=O)O)NC(=O)[C@H](CCCNC(=N)N)NC(=O)[C@H](CC(=O)O)NC(=O)[C@H](CCCCn3cc(CCCC(=O)Nc4nnc(S(N)(=O)=O)s4)nn3)NC(=O)C[C@H](NC(=O)C[C@H](NC(=O)CCC(C)(c3ccc(O)cc3)c3ccc(O)cc3)C(=O)O)C(=O)O)C(=O)O)C2=O)C(C)C)cc1)C(C)C]

Chemical Formula: C₁₂₄H₁₈₀N₂₈O₃₇S₃

### Molecular Weight: 2751.14

Compound 1 (3.5 mg, 2.44 mol, 1.1 eq) was dissolved in degassed PBS (pH 7.4; 600 μl). Commercially available Maleimidocaproyl-ValCit-p-aminobenzylalcohol-MMAE (3.0 mg, 2.23 mol, 1.0 eq) was added as a DMF solution (500 μl) and the mixture was stirred at room temperature. After 3 hours, UPLC-MS indicated completion. See FIG. 1B, which demonstrates the analytical UPLC trace of compound 2 on a BEH C18 Column, 130 Å, 1.7 μm, 2.1 mm×50 mm at a flow rate of 0.6 ml min⁻¹, 5% MeCN in 0.1% aq. FA to 80% MeCN in 6 min.

The solvents were removed under vacuum and the crude was diluted in a 1:1 H₂O/MeCN mixture (1 ml). The mixture was purified by RP-HPLC (Synergi RP Polar, 5% MeCN in 0.1% aq. TFA to 80% over 20 min). Pure fractions were collected and lyophilized overnight to achieve 2 as a white solid (3.1 mg; 1.13 mol; 50.8% yield).

MS (ESI) m/z calcd. for [C₁₂₄H₁₈₂N₂₈O₃₇S₃]²⁺: 1375.6113 [M+2H]²⁺, found: 1375.6986; m/z calcd. for [C₁₂₄H₁₈₃N₂₈O₃₇S₃]³⁺: 917.4075 [M+3H]³±, found: 917.4496.

### Cell Cultures

A sample of renal cell carcinoma cell line SKRC-52 was thawed and cells were kept in culture in RPMI medium (Invitrogen) supplemented with fetal calf serum (10%, FCS, Invitrogen) and Antibiotic-Antimycotic (1%, AA, Invitrogen) at 37° C. and 5% CO₂. For passaging, cells were detached using Trypsin-EDTA 0.05% (Invitrogen) when reaching 90% confluence and re-seeded at a dilution of 1:6.

### Animal Studies

All animal experiments were conducted in accordance with Swiss animal welfare laws and regulations under the license number 27/2015 granted by the Veterinäramt des Kantons Zurich.

### Implantation of Subcutaneous SKRC-52 Tumors

SKRC-52 cells were grown to 80% confluence and detached with Trypsin-EDTA 0.05% (Life Technologies). Cells were washed with Hank's Balanced Salt Solution (HBSS, pH 7.4) once, counted and re-suspended in HBSS to a final concentration of 5×10⁷ cells/ml. Aliquots of 5×10⁶ cells (100 μl of a suspension) were injected subcutaneously in the right flank of female athymic BALB/c nu/nu mice (6-8 weeks of age, Janvier).

### Example 2: Tumor Therapy Experiment Using CAIX Ligand Armed with MMAE in Combination with L19-IL2

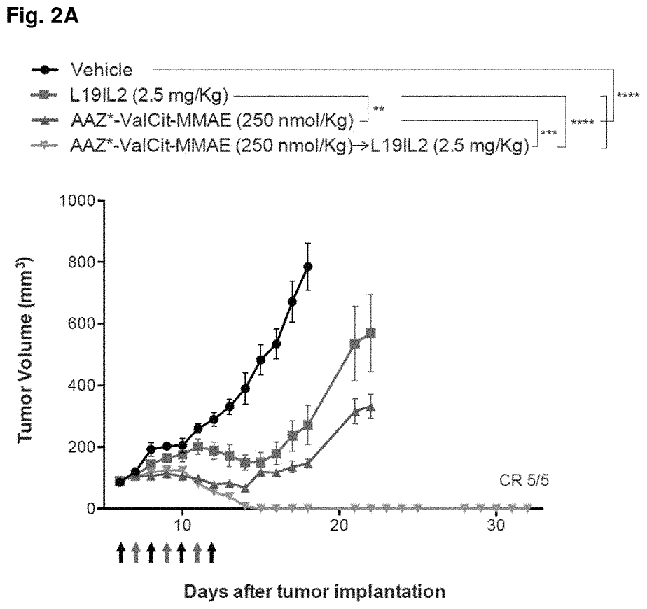

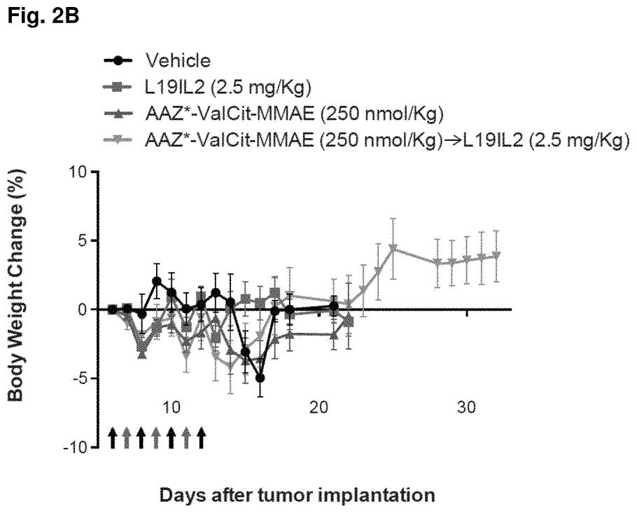

SKRC-52 xenografted tumors were implanted into female BALB/c nu/nu mice (Janvier) as described above, and allowed to grow to an average volume of 0.1 ml. Mice were randomly assigned into therapy groups of 5 animals and treatment started. Treatment consisted in daily injections (IV, tail vein) of compound 2 (dissolved in PBS containing 1% of DMSO, see example 1) at the dose of 250 nmol/Kg (determined as Maximum Tolerated Dose, MTD, in nude mice; data not shown), alternated with L19-IL2 at the dose of 2.5 mg/Kg (schedule depicted in FIGS. 2A and 2B). Control groups were treated with PBS (containing 1% of DMSO), compound 2 alone (250 nmol/Kg), or L19-IL2 (2.5 mg/Kg) alone. Animals were weighed (see FIG. 2B) and tumor sizes were measured daily with an electronic caliper. The tumor volume was calculated according to the formula (long side)×(short side)×(short side)×0.5 (see FIG. 2A). Animals were sacrificed when the termination criteria were reached. Prism 6 software (GraphPad Software) was used for data analysis (regular two-way ANOVA with the Bonferroni test).

As can be seen in FIG. 2A and FIG. 3, by day 17, 5 out of 5 of the xenografted tumors completely regressed with a CAIX ligand armed with MMAE in combination with L19-IL2. Remarkably, no tumor lesions were visible or palpable for several days thereafter, indicating that the regression can be regarded as a total cure for all mice treated. This surprising outcome could not have been predicted from the effects of the individual components.

### Example 3: Tumor Rechallenge Study Using CAIX Ligand Armed with MMAE in Combination with L19-IL2

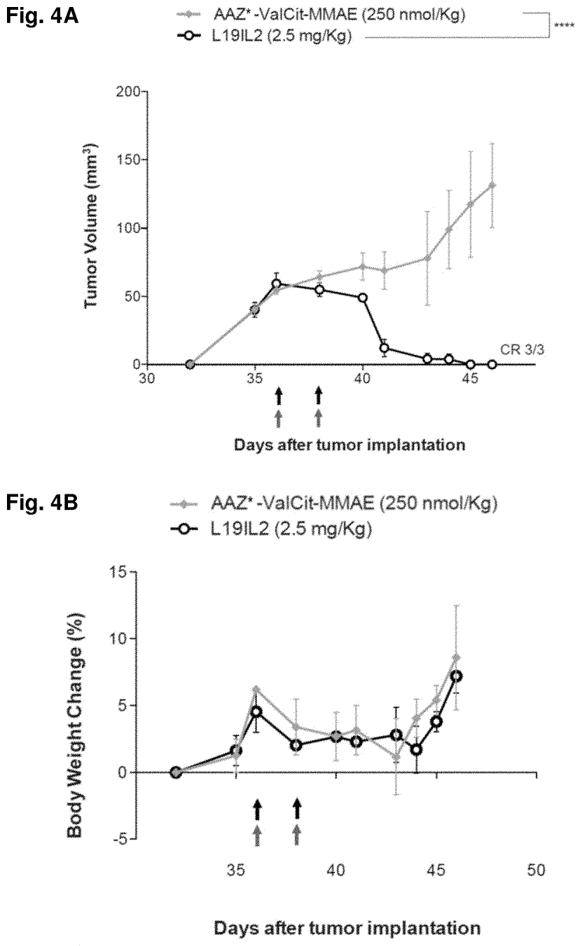

To determine whether the CAIX ligand armed with MMAE (compound 2, see example 1) in combination with L19-IL2 caused acquired protective immunity against the tumor, mice that were cured in the therapy experiment of Example 2 were injected with 5×10⁶ cells (100 μl of a suspension) SKRC-52 cells/mouse 20 days after the treatment ended. All the tumors were growing back after the rechallenge. Mice were rearranged in two groups and treated with either the compound 2 alone (250 nmol/Kg; 2 mice), or L19-IL2 (2.5 mg/Kg; 3 mice) alone (see the schedule in FIGS. 4A and 4B). Tumor volume was determined as in the therapy experiments according to the formula (long side)×(short side)×(short side)×0.5. Only the treatment with the L19-IL2 immunocytokine induces a second complete tumor regression for all 3 animals composing the treatment group.

### Example 4: Tumor Therapy Experiment Using CAIX Ligand Armed with MMAE in Combination with F8-IL2

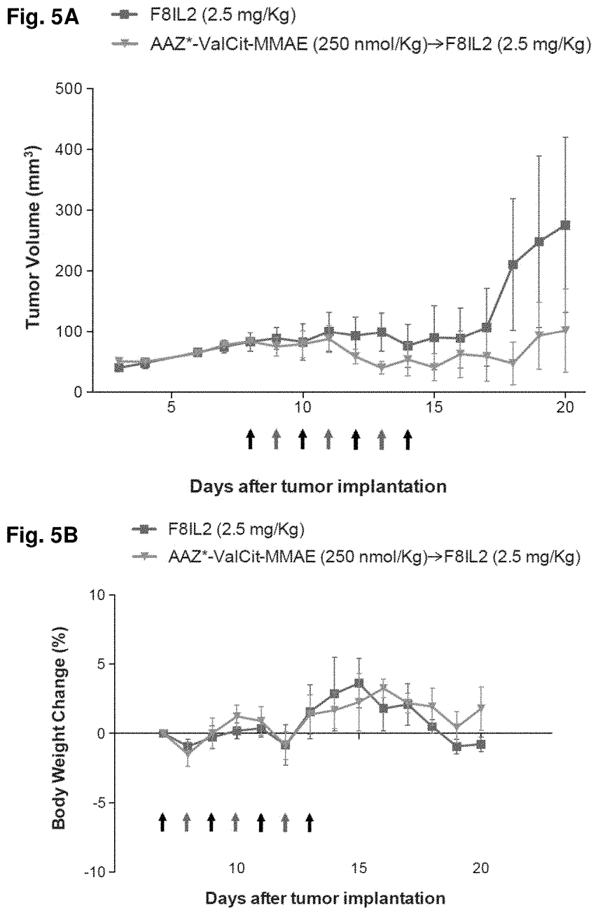

SKRC-52 xenografted tumors were implanted into female BALB/c nu/nu mice (Janvier) as described above, and allowed to grow to an average volume of 0.1 ml. Mice were randomly assigned into therapy groups of 3 animals and treatment started. Treatment consisted in daily injections (IV, tail vein) of compound 2 (dissolved in PBS containing 1% of DMSO, see Example 1) at the dose of 250 nmol/Kg, alternated with F8IL2 (produced by a stable cell line) at the dose of 2.5 mg/Kg (schedule depicted in FIGS. 5A and 5B). Control groups were treated with F8-IL2 only at the dose of 2.5 mg/Kg. Animals were weighed (see FIG. 5B) and tumor sizes were measured daily with an electronic caliper. The tumor volume was calculated according to the formula (long side)×(short side)×(short side)×0.5. Animals were sacrificed when the termination criteria were reached. Prism 6 software (GraphPad Software) was used for data analysis (regular two-way ANOVA with the Bonferroni test).

As can be seen in FIG. 5A, a better control of the tumor growth was achieved, but no complete responses were observed for the combination group compared to the control group.

### Example 5: Tumor Therapy Experiment Using CAIX Ligand Armed with MMAE in Combination with L19-IL2 in a Murine Model of Colorectal Cancer

### Transfection of Human CAIX in CT26 Tumor Cells and Monoclonal Selection

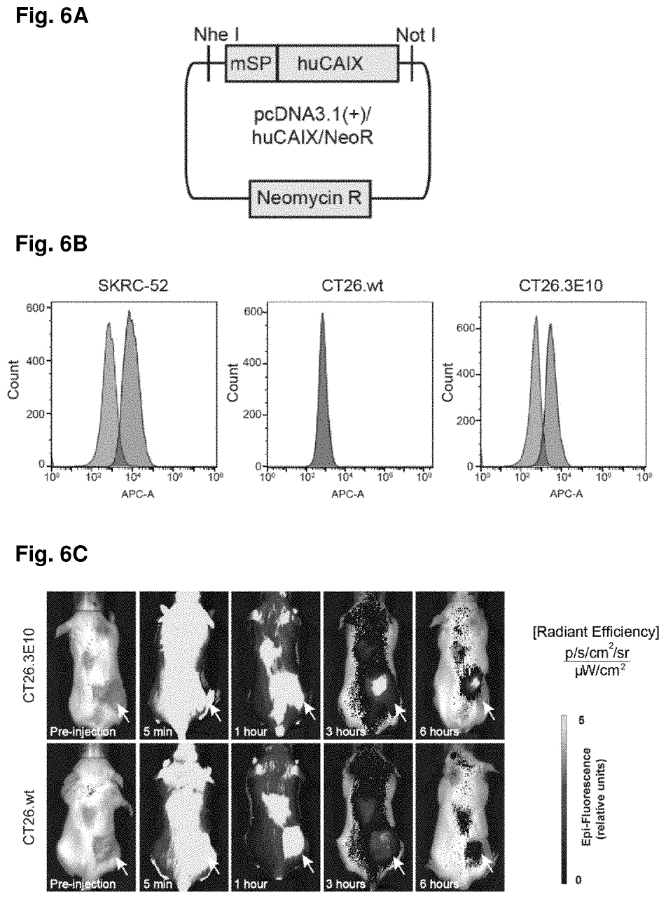

The gene for human CAIX was cloned into the mammalian expression vector pcDNA3.1(+) (FIG. 6A) containing an antibiotic resistance for G418 Geneticin. 6×10⁷ CT26.wt cells were transfected with 60 μg of pcDNA3.1(+) containing the human CAIX gene using the Amaxa™ 4 D-Nucleofector (Lonza) with the SG Cell Line 4D-Nucleofector® X Kit L (Lonza) and re-seeded in complete growing medium. Three days after the transfection, the medium was replaced with RPMI (10% FCS, 1% AA) containing 0.5 mg/ml G418 (Merck) to select a stably transfected polyclonal cell line. To yield a monoclonal cell line, the stable cell line was stained as described for FACS analysis and single cell sorting was performed using a BD FACSAria III. Different clones were expanded and checked for antigen expression. Clone CT26.3E10 was selected for CAIX expression by FACS and immunofluorescence microscopy, and used for further in vivo experiments.

### Cell Cultures

The murine colorectal carcinoma cell line CT26.wt (ATCC) was maintained in RPMI medium (Invitrogen) supplemented with fetal calf serum (10%, FCS, Invitrogen) and Antibiotic-Antimycotic (1%, AA, Invitrogen) and cultured at 37° C. and 5% CO₂ For passaging, confluent cells were detached using Trypsin-EDTA 0.05% (Invitrogen) and re-seeded at a dilution of 1:5. Transfected CT26 cells were kept in the same culture conditions as CT26.wt cells.

### FACS Analysis

For cellular expression analysis of human CAIX, cells were detached with 50 mM EDTA and 5×10⁵ cells were stained with an anti-CAIX specific antibody (500 nM, 1 hour, 4° C.) in a volume of 100 μl FACS-Buffer (0.5% BSA, 2 mM EDTA in PBS). For signal amplification, the anti-CAIX antibody was detected with an anti-human AlexaFluor647 labeled antibody (Invitrogen) (1:200, 45 min, 4° C.). In-between and after staining, cells were washed with 100 μl FACS-Buffer and centrifuged at 1100 rpm for 3 min Stained cells were analyzed with a 2 L-Cytoflex (Beckman-Coulter). SKRC-52 cells were used as positive control and CT26.wt cells as negative control. Results were analyzed with FlowJo 9 (FlowJo LLC).

### Animal Studies

All animal experiments were conducted in accordance with Swiss animal welfare laws and regulations under the license number 27/2015 granted by the Veterinäramt des Kantons Zurich.

### Implantation of Subcutaneous Tumors

CT26.3E10 cells were grown to 80% confluence and detached with Trypsin-EDTA 0.05% (Life Technologies). Cells were washed with Hank's Balanced Salt Solution (HBSS, pH 7.4) once, counted and re-suspended in HBSS to a final concentration of 6.0×10⁷ cells ml⁻¹. Aliquots of 6×10⁶ cells (100 μl of the suspension) were injected subcutaneously in the right flank of female BALB/c mice (8-10 weeks of age, Janvier).

### In Vivo Imaging System (IVIS): Near Infrared Fluorescence Imaging Evaluation

Female BALB/c mice bearing subcutaneous CT26.3E10 or CT26.wt tumors were injected intravenously with acetazolamide labeled with the near infrared dye moiety IRDye680RD (AAZ-IRDye680RD; compound 3; 250 nmol/Kg), dissolved in sterile PBS (100 μl). Mice were anesthetized with isoflurane and fluorescence images acquired on an IVIS Spectrum imaging system (Xenogen, exposure 1 s, binning factor 8, excitation at 675 nm, emission filter at 720 nm, f number 2, field of view 13.1). Images were taken before the injection and after 5 min, 1 hour, 3 hours and 6 hours. Food and water were given ad libitum during that period. Mice were sacrificed after the last picture by cervical dislocation.

### Synthesis of AAZ-IRDye680RD (Compound 3)

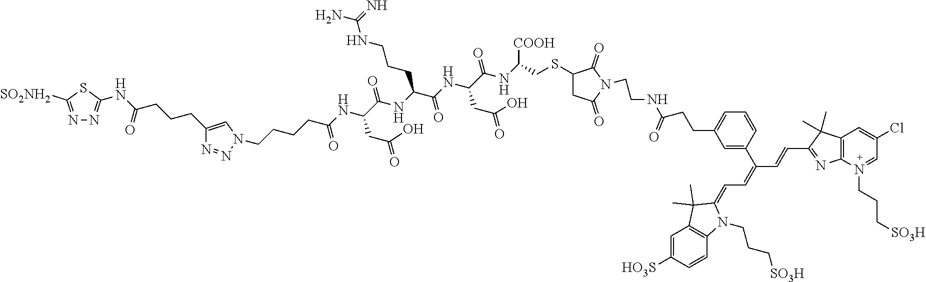

Chemical Formula: C₇₅H₉₇ClN₁₉O₂₅S₆

### Molecular Weight: 1892.52

Compound 3 was prepared according to previously described procedures (Cazzamalli S, Dal Corso A, Neri D. Acetazolamide serves as selective delivery vehicle for dipeptide-linked drugs to renal cell carcinoma. Mol Cancer Ther 2016).

### Therapy Experiments

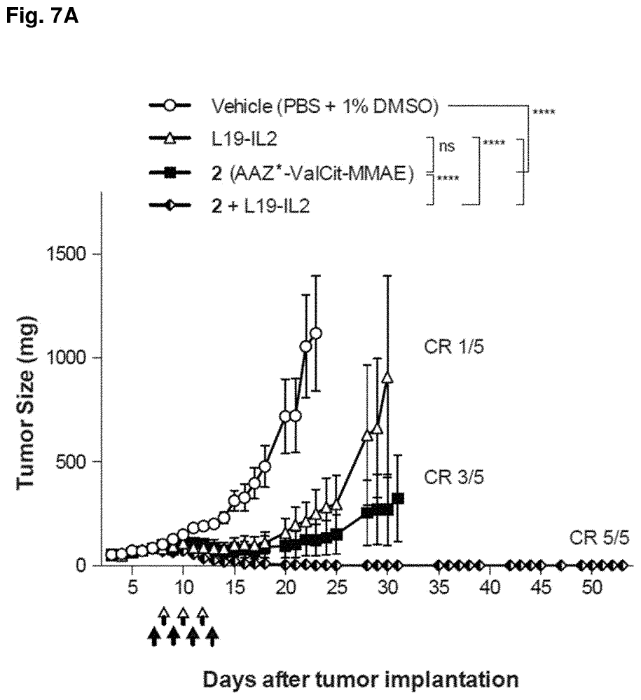

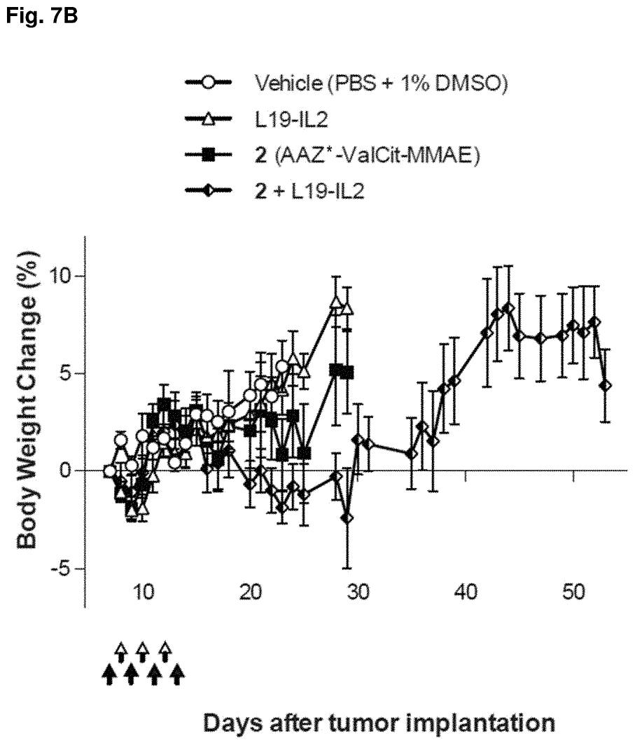

CT26.3E10 tumors was implanted into female BALB/c mice (Janvier) as described above, and allowed to grow to an average volume of 100 mm³. Mice were randomly assigned into therapy groups of 4 or 5 animals and treatment started by injecting a solution of AAZ*-ValCit-MMAE (compound 2, 250 nmol/Kg), L19-IL2 (2.5 mg/Kg), combination or vehicle (PBS containing 1% of DMSO) intravenously (lateral tail vein) at the doses and with the schedules depicted in FIG. 7.

Compound 2 was injected as solutions in sterile PBS containing 1% DMSO. L19-IL2 was injected as solution in appropriate sterile formulation buffer (Philogen). Animals were weighed and tumor sizes measured daily with an electronic caliper. The tumor volume was calculated according to the formula (long side)×(short side)×(short side)×0.5. Animals were sacrificed when the termination criteria were reached. Prism 6 software (GraphPad Software) was used for data analysis (regular two-way ANOVA followed by Bonferroni test).

### Results of the Therapy Experiments in Immunocompetent Mice, Bearing Syngeneic CT26.3E10 Tumors

Approximately 30% of patients with colorectal cancer express high levels of CAIX in their tumor, but the immunocompetent mouse models of the disease tested by the inventors were negative for the antigen (data not shown). In order to establish an immunocompetent murine model of colorectal cancer expressing CAIX, the inventors stably-transfected colorectal CT26.wt cancer cells with a gene coding for human CAIX and a second gene conferring antibiotic resistance against Neomycin (FIG. 6A). After antibiotic selection and single cell sorting, monoclonal cell lines were tested for CAIX expression. Clone CT26.3E10 (but not CT26.wt) showed a shift in fluorescence intensity in FACS analysis using an anti-CAIX monoclonal antibody. The FACS shift was similar to the one observed for SKRC-52 cells (FIG. 6B). Fluorescence microscopy experiments confirmed CAIX expression on the membrane of CT26.3E10 and SKRC-52 cells, but not in wild type CT26 cells (data not shown). Furthermore, mice bearing CT26.3E10 tumors showed a preferential uptake of the near-infrared fluorophore conjugate AAZ-IRdye680RD in the neoplastic mass 6 hours after intravenous injection, compared to similar experiments performed in mice bearing CT26.wt tumors (FIG. 6C).

Therapy experiments were then performed using the CAIX ligand armed with MMAE (compound 2, see Example 1), alone or in combination with the L19-IL2, in immunocompetent BALB/c mice bearing subcutaneous CT26.3E10 tumors. Also in this case, the antitumor effect was strongest when both therapeutic agents were used in combination (p<0.0001), leading to durable complete responses in 100% of treated mice (FIG. 7).

### Example 6: Comparative Therapy Experiments Between AAZ*-ValCit-MMAE (Compound 2) and AAZ-ValCit-MMAE (Compound 4) in SKRC-52 Renal Cell Carcinoma Model

### Synthesis of AAZ-ValCit-MMAE (Compound 4)

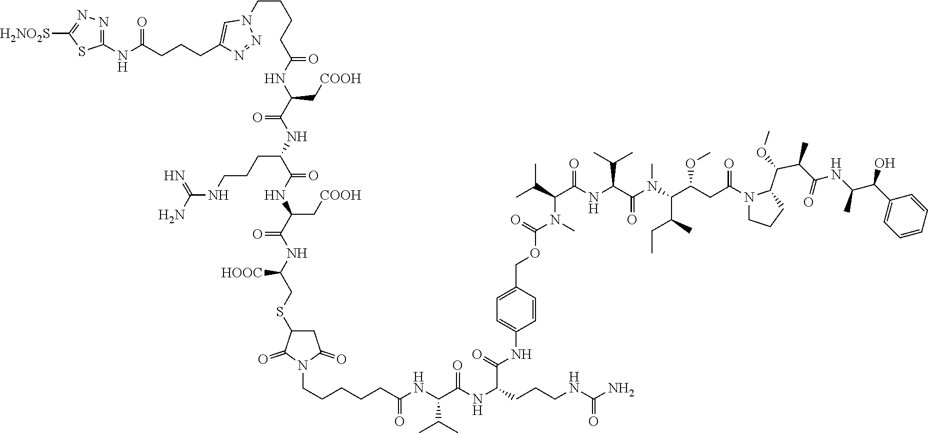 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC[C@H](NC(=O)[C@H](CC(=O)O)NC(=O)[C@H](CCCNC(=N)N)NC(=O)[C@H](CC(=O)O)NC(=O)CCCCn3cc(CCCC(=O)Nc4nnc(S(N)(=O)=O)s4)nn3)C(=O)O)C2=O)C(C)C)cc1)C(C)C]

Chemical Formula: C₉₈H₁₅₁N₂₅O₂₈S₃

### Molecular Weight: 2223.61

Compound 4 was prepared according to previously described procedures (Cazzamalli S, Dal Corso A, Neri D. Acetazolamide serves as selective delivery vehicle for dipeptide-linked drugs to renal cell carcinoma. Mol Cancer Ther 2016 and Cazzamalli S, Corso A D, Neri D. Linker stability influences the anti-tumor activity of acetazolamide-drug conjugates for the therapy of renal cell carcinoma. J Control Release 2017; 246:39-45).

Synthesis of NH₂⁺-ValCit-MMAE (Negative Control Precursor; Compound 5) in S Configuration

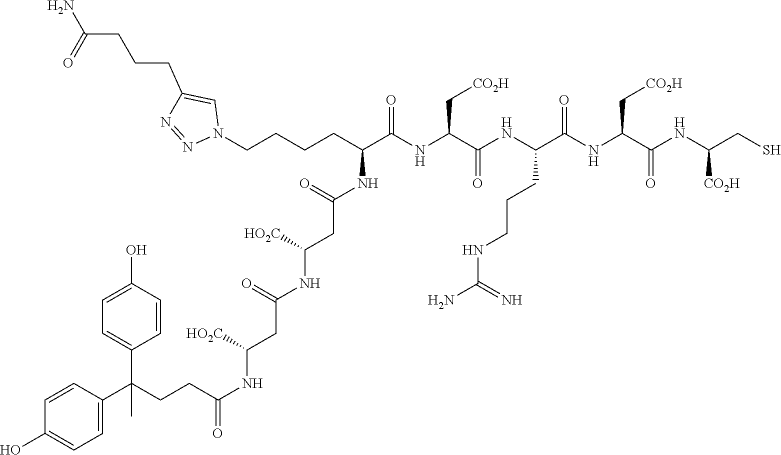 [SMILES: CC(CCC(=O)N[C@@H](CC(=O)N[C@@H](CC(=O)N[C@@H](CCCCn1cc(CCCC(N)=O)nn1)C(=O)N[C@@H](CC(=O)O)C(=O)N[C@@H](CCCNC(=N)N)C(=O)N[C@@H](CC(=O)O)C(=O)N[C@@H](CS)C(=O)O)C(=O)O)C(=O)O)(c1ccc(O)cc1)c1ccc(O)cc1]

Chemical Formula: C₅₄H₇₄N₁₄O₂₀S

### Molecular Weight: 1271.32

Commercially available pre-loaded Fmoc-Cys(Trt)-OH on polystyrene resin (300 mg, 0.19 mmol) was swollen in DMF for 15 min. The Fmoc group was removed with 20% piperidine in DMF (3×10 min×10 ml) and the resin washed with DMF (4×10 min×10 ml). Fmoc-Asp(OtBu)-OH (233 mg, 0.57 mmol, 3 eq) was activated with HATU (215 mg, 0.57 mmol, 3 eq), and DIPEA (197 μl, 1.13 mmol, 6 eq) in DMF (3 ml) at 0° C. for 15 min and then reacted with the resin for 1 h under gentle agitation. After washing the resin with DMF (4×10 min×10 ml) the Fmoc group was removed with 20% piperidine in DMF (3×10 min×10 ml) and the resin washed with DMF (4×10 min×10 ml) before the peptide was extended with Fmoc-Arg(Pbf)-OH (368 mg, 0.57 mmol, 3 eq), Fmoc-Asp(OtBu)-OH (233 mg, 0.57 mmol, 3 eq), Fmoc-Lys(N3)-OH (224 mg, 0.57 mmol, 3 eq), Fmoc-Asp-OtBu (233, 0.57 mmol, 3 eq), Fmoc-Asp-OtBu (233, 0.57 mmol, 3 eq) and 4,4-bis(4-hydroxyphenyl)valeric acid (162 mg, 0.57 eq, 3 eq) in the indicated order using the same coupling conditions (HATU/DIPEA), Fmoc-deprotection (20% piperidine in DMF) and washing step with DMF mentioned before. After the last peptide-coupling step, a solution of CuI (11 mg, 0.06 mmol, 0.3 eq), TBTA (10 mg, 0.02 mmol, 0.1 eq) and Hex-5-ynamide (63 mg, 0.57 mmol, 3 eq) in a mixture of DMF (1.5 ml) and THF (1.5 ml) was prepared and reacted with the resin at room temperature for 48 h. After washing with DMF (4×10 min×10 ml), EDTA 50 mM (4×10 min×10 ml) and DCM (4×10 min×10 ml), the compound was cleaved from the resin by agitating with a mixture of TFA (6 ml), TIS (1.1 ml), H₂O (300 μl), m-Cresol (300 μl) and Thioanisol (300 μl) at room temperature for 1 h. Cleavage solution was added drop-wise to ice cold diethyl ether (50 ml) obtaining a white precipitate. The pellet was collected by centrifugation, dried under vacuum, redissolved in Millipore water and added with an excess of Tris(2-carboxyethyl)phosphine hydrochloride (30 eq). The product was purified by reversed-phase HPLC (Synergi RP Polar, 5% MeCN in 0.1% aq. TFA to 80% over 20 min). After lyophilization the final compound was collected as a white powder (27 mg, 21.2 μmol, 11% yield).

MS (ESI) m/z calcd. for [C₅₄H₇₅N₁₄O₂₀S]¹⁺: 1271.4924 [M+H]¹⁺, found: 1271.5669; m/z calcd. for [C₅₄H₇₆N₁₄O₂₀S]²⁺: 636.2462 [M+2H]²⁺, found: 636.2623.
Synthesis of NH₂⁺-ValCit-MMAE Negative Control (Compound 6) in S Configuration

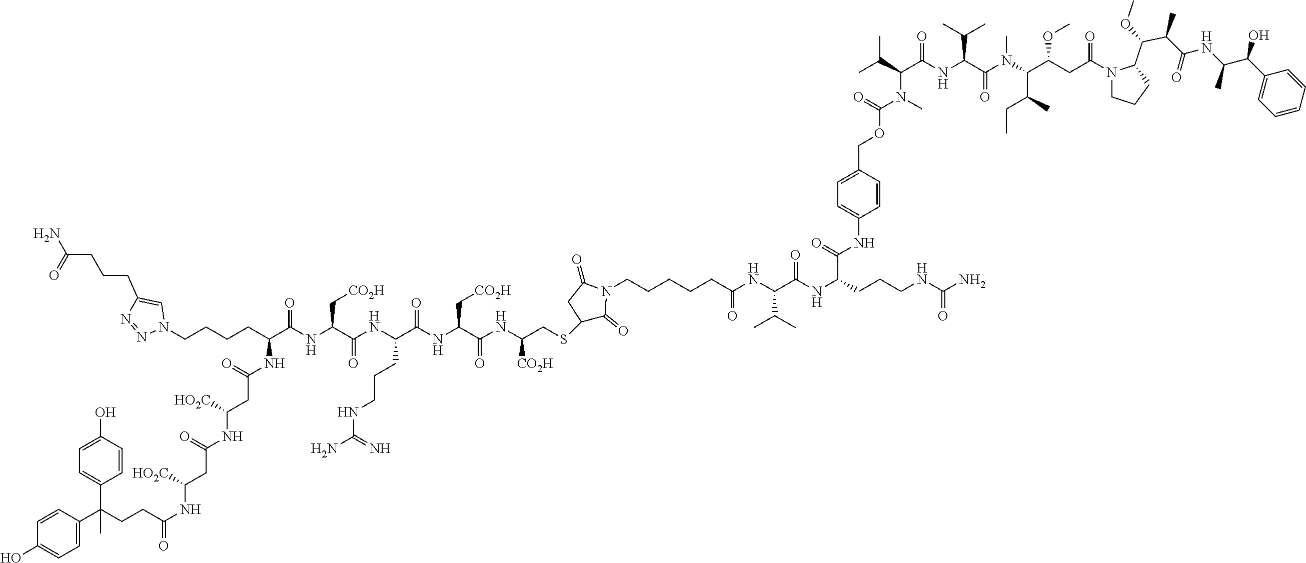 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC[C@H](NC(=O)[C@H](CC(=O)O)NC(=O)[C@H](CCCNC(=N)N)NC(=O)[C@H](CC(=O)O)NC(=O)[C@H](CCCCn3cc(CCCC(N)=O)nn3)NC(=O)C[C@H](NC(=O)C[C@H](NC(=O)CCC(C)(c3ccc(O)cc3)c3ccc(O)cc3)C(=O)O)C(=O)O)C(=O)O)C2=O)C(C)C)cc1)C(C)C]

Chemical Formula: C₁₂₂H₁₇₉N₂₅O₃₅S₁

### Molecular Weight: 2587.97

Compound 5 (2.5 mg, 1.97 μmol, 1.3 eq) was dissolved in degassed PBS (pH 7.4; 600 μl). Commercially available Maleimidocaproyl-ValCit-p-aminobenzylalcohol-MMAE (2.0 mg, 1.52 μmol, 1.0 eq) was added as a DMF solution (500 μl) and the mixture was stirred at room temperature. After 3 hours, UPLC-MS indicated completion. The solvents were removed under vacuum and the crude was diluted in a 1:1 H2O/MeCN mixture (1 ml). The mixture was purified by RP-HPLC (Synergi RP Polar, 5% MeCN in 0.1% aq. TFA to 80% over 20 min). Pure fractions were collected and lyophilized overnight to achieve 6 as a white solid (2.2 mg; 0.85 μmol; 56.4% yield).
MS (ESI) m/z calcd. for [C₁₂₂H₁₈₁N₂₅O₃₅S]²⁺: 1294.1358 [M+2H]²⁺, found: 1294.2177; m/z calcd. for [C₁₂₂H₁₈₂N₂₅O₃₅S]³⁺: 863.0905 [M+3H]³⁺, found: 863.1285.

### Synthesis of AAZ*-NH2 Ligand (Compound 7) in S Configuration

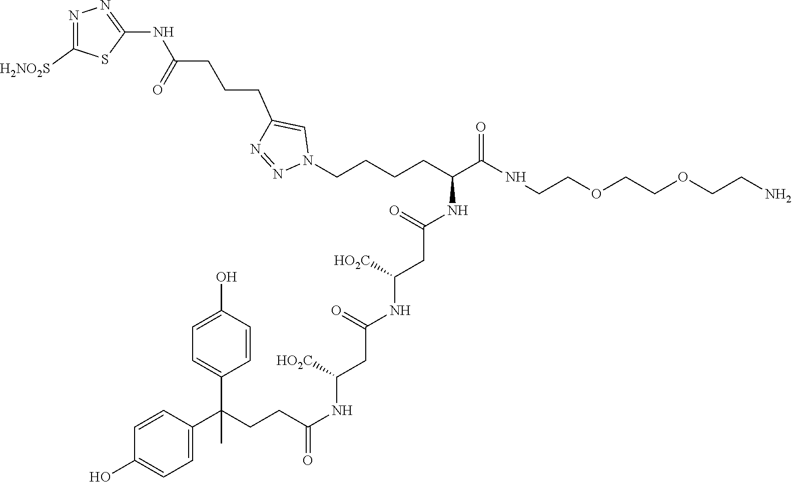 [SMILES: CC(CCC(=O)N[C@@H](CC(=O)N[C@@H](CC(=O)N[C@@H](CCCCn1cc(CCCC(=O)Nc2nnc(S(N)(=O)=O)s2)nn1)C(=O)NCCOCCOCCN)C(=O)O)C(=O)O)(c1ccc(O)cc1)c1ccc(O)cc1]

Chemical Formula: C₄₅H₆₂N₁₂O₁₅S₂

### Molecular Weight: 1075.18

Compound 7 was prepared according to previously described procedures (Wichert M, Krall N, Decurtins W, Franzini R M, Pretto F, Schneider P, et al. Dual-display of small molecules enables the discovery of ligand pairs and facilitates affinity maturation. Nat Chem 2015; 7(3):241-9) or WO2015/114171.

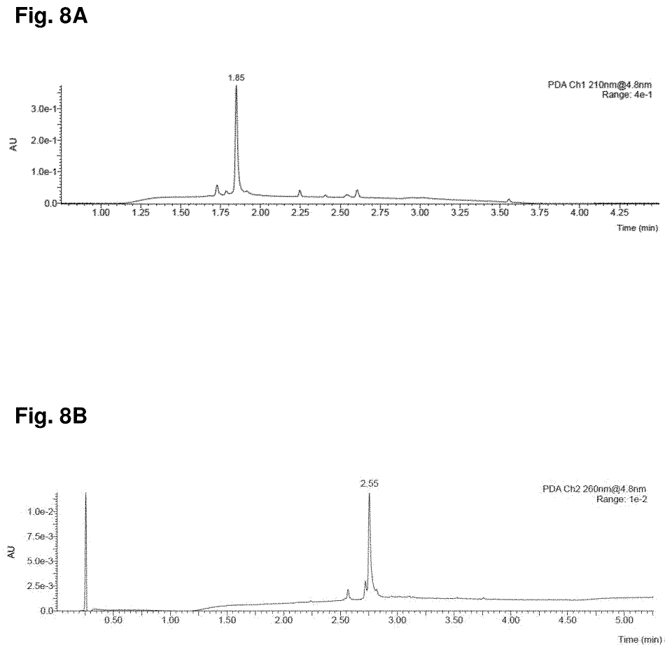

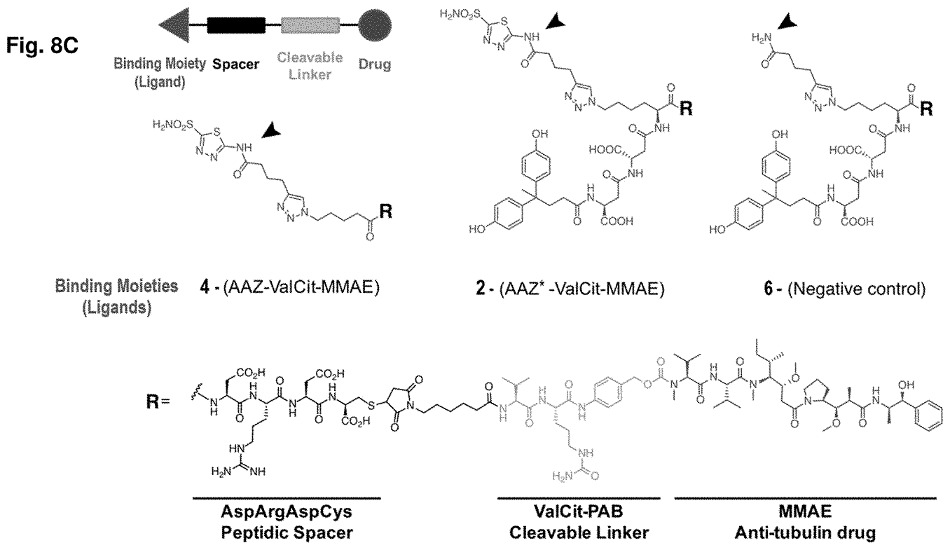

The resulting SMDCs (compound 2) and (compound 4) and the negative control (compound 6) (FIG. 8C) were obtained as highly pure (>95% purity by UPLC) lyophilized material (see FIG. 1B and FIG. 8B for SMDC 2 and negative control respectively) and used for the subsequent therapy experiments in SKRC-52 renal cell carcinoma model.

### Anticancer Activity of Compounds AAZ*-ValCit-MMAE (2) and AAZ-ValCit-MMAE (4) in BALB/c Nude Mice, Bearing Subcutaneously-Grafted SKRC-52 Renal Cell Carcinomas.

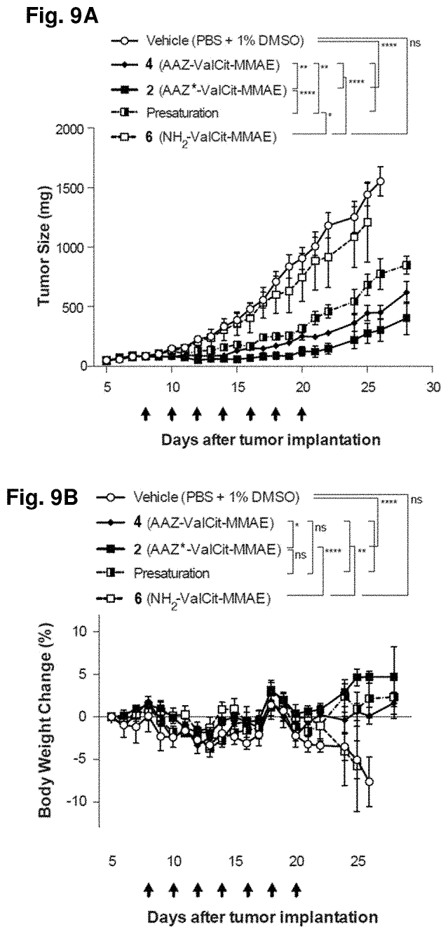

Compound 6, devoid of the CAIX-targeting moiety but otherwise identical to compound 2, served as negative control (FIG. 8C). The SMDCs were administered at the maximum tolerated dose, which had been found to be 250 nmol/Kg in preliminary experiments (data not shown) following the schedule depicted in FIG. 9A. A fourth group of mice (“presaturation” group) was injected with a 50-fold higher dose (12.5 μmol/Kg) of ligand AAZ* (compound 7), which was directly followed by the administration of AAZ*-ValCit-MMAE (compound 2). Both compounds 2 and 4 showed a potent anti-cancer activity (p<0.0001 compared to the group of mice treated with vehicle), while negative control compound 6 did not display a difference from the vehicle-treated group. Compound 2 was very well tolerated (FIG. 9B) and exhibited the most potent tumor growth retardation effect, but no mice had a durable complete response.

### Example 7: Quantitative and Qualitative Biodistribution of AAZ* and of AAZ in the SKRC-52 Model

### Synthesis of the AAZ*-⁹⁹mTc Chelator (Compound 8) in S Configuration

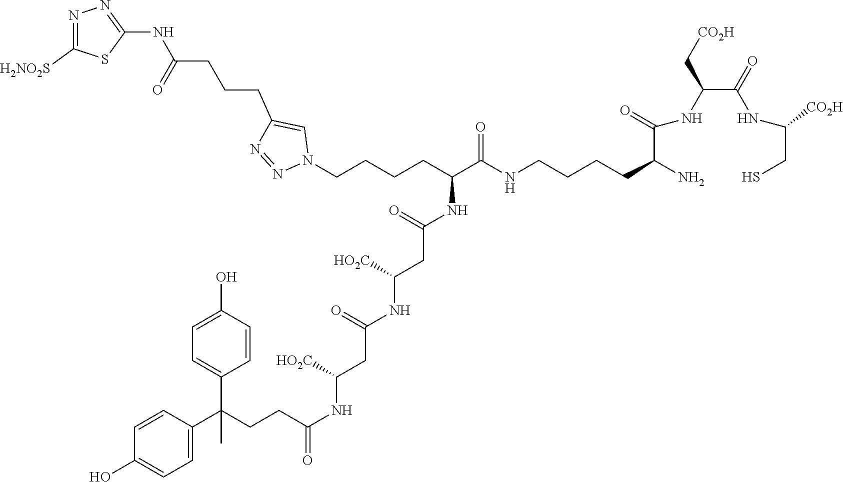 [SMILES: CC(CCC(=O)N[C@@H](CC(=O)N[C@@H](CC(=O)N[C@@H](CCCCn1cc(CCCC(=O)Nc2nnc(S(N)(=O)=O)s2)nn1)C(=O)NCCCC[C@H](N)C(=O)N[C@@H](CC(=O)O)C(=O)N[C@@H](CS)C(=O)O)C(=O)O)C(=O)O)(c1ccc(O)cc1)c1ccc(O)cc1]

Chemical Formula: C₅₂H₇₀N₁₄O₁₉S₃

### Molecular Weight: 1291.39

Commercially available pre-loaded Fmoc-Cys(Trt)-OH on polystyrene resin (500 mg, 0.32 mmol) was swollen in DMF for 15 min. The Fmoc group was removed with 20% piperidine in DMF (3×10 min×10 ml) and the resin washed with DMF (4×10 min×10 ml). Fmoc-Asp(OtBu)-OH (388 mg, 0.95 mmol, 3 eq) was activated with HATU (358 mg, 0.95 mmol, 3 eq), and DIPEA (328 μl, 1.88 mmol, 6 eq) in DMF (5 ml) at 0° C. for 5 min and then reacted with the resin for 1 h under gentle agitation. After washing the resin with DMF (4×10 min×10 ml) the Fmoc group was removed with 20% piperidine in DMF (3×10 min×10 ml) and the resin washed with DMF (4×10 min×10 ml) before the peptide was extended with Boc-Lys(Fmoc)-OH (445 mg, 0.95 mmol, 3 eq), Fmoc-Lys(N3)-OH (374 mg, 0.95 mmol, 3 eq), Fmoc-Asp(OH)-OtBu (388 mg, 0.95 mmol, 3 eq), Fmoc-Asp(OH)-OtBu (388 mg, 0.95 mmol, 3 eq) and 4,4-bis(4-hydroxyphenyl)valeric acid (162 mg, 0.95 eq, 3 eq) in the indicated order using the same coupling conditions (HATU/DIPEA), Fmoc-deprotection (20% piperidine in DMF) and washing step with DMF mentioned before.

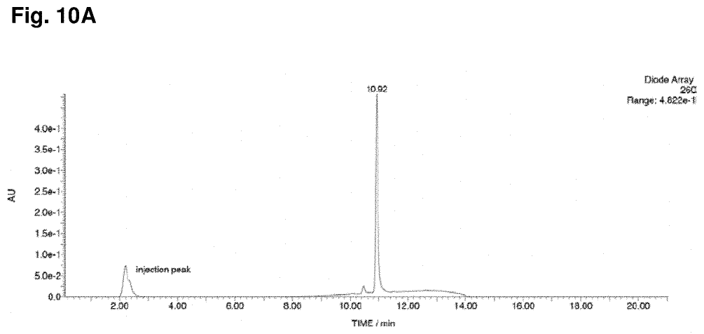

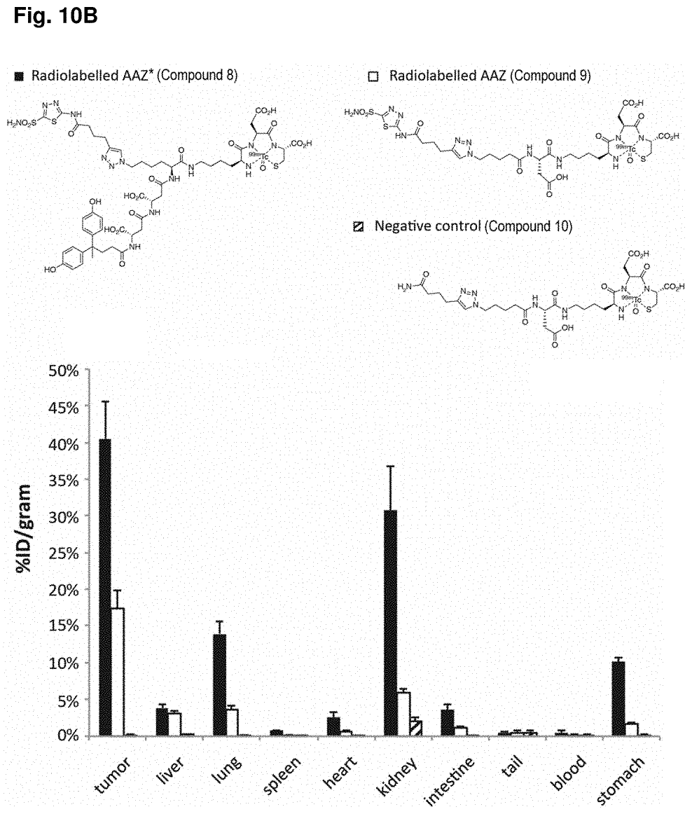

After the last peptide-coupling step, a solution of CuI (18 mg, 0.095 mmol, 0.3 eq), TBTA (16 mg, 0.031 mmol, 0.1 eq) and N-(5-sulfamoyl-1,3,4-thiadiazol-2-yl)hex-5-ynamide (261 mg, 0.95 mmol, 3 eq) in a mixture of DMF (2.5 ml) and THF (2.5 ml) was prepared and reacted with the resin at room temperature for 48 h. After washing with DMF (4×10 min×10 ml), EDTA 50 min (4×10 min×10 ml) and DCM (4×10 min×10 ml), the compound was cleaved from the resin by agitating with a mixture of TFA (8.6 ml), TIS (1.6 ml), H₂O (400 μl), m-Cresol (400 μl) and Thioanisol (400 μl) at room temperature for 1 h. Cleavage solution was added drop-wise to ice cold diethyl ether (50 ml) obtaining a white precipitate. The pellet was collected by centrifugation, dried under vacuum, redissolved in Millipore water and added with an excess of Tris(2-carboxyethyl)phosphine hydrochloride (30 eq). The product was purified by reversed-phase HPLC (Synergi RP Polar, 5% MeCN in 0.1% aq. TFA to 80% over 20 min). The analytical HPLC trace of compound 8 is shown in FIG. 10A. The injection peak at around 2 minutes is an artefact. After lyophilization the final compound was collected as a white powder (91 mg, 70.5 μmol, 22% yield).

MS (ESI) m/z calcd. for [C₅₂H₇₁N₁₄O₁₉S₃]¹⁺: 1291.4104 [M+H]¹⁺, found: 1291.4219.

### Synthesis of the AAZ-⁹⁹mTc Chelator (Compound 9)

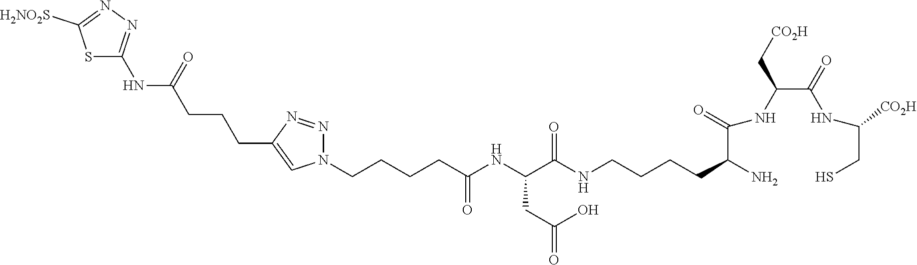 [SMILES: N[C@@H](CCCCNC(=O)[C@H](CC(=O)O)NC(=O)CCCCn1cc(CCCC(=O)Nc2nnc(S(N)(=O)=O)s2)nn1)C(=O)N[C@@H](CC(=O)O)C(=O)N[C@@H](CS)C(=O)O]

Chemical Formula: C₃₀H₄₆N₁₂O₁₃S₃

### Molecular Weight: 878.95

Compound 9 was prepared according to previously described procedures (Cazzamalli S, Dal Corso A, Neri D. Acetazolamide serves as selective delivery vehicle for dipeptide-linked drugs to renal cell carcinoma. Mol Cancer Ther 2016 and Krall N, Pretto F, Mattarella M, Muller C, Neri D. A technetium 99m-labeled ligand of carbonic anhydrase IX selectively targets renal cell carcinoma in vivo. J Nucl Med 2016).

Synthesis of the NH₂-⁹⁹mTc Chelator (Negative Control; Compound 10)

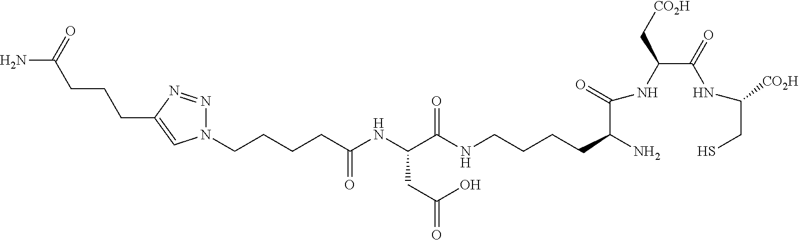 [SMILES: NC(=O)CCCc1cn(CCCCC(=O)N[C@@H](CC(=O)O)C(=O)NCCCC[C@H](N)C(=O)N[C@@H](CC(=O)O)C(=O)N[C@@H](CS)C(=O)O)nn1]

Chemical Formula: C₂₈H₄₅N₉O₁₁S

### Molecular Weight: 715.78

Compound 10 was prepared according to previously described procedures (Krall N, Pretto F, Mattarella M, Muller C, Neri D. A technetium 99m-labeled ligand of carbonic anhydrase IX selectively targets renal cell carcinoma in vivo. J Nucl Med 2016).

### Synthesis of the AAZ*-IRDye680RD (Compound 11)

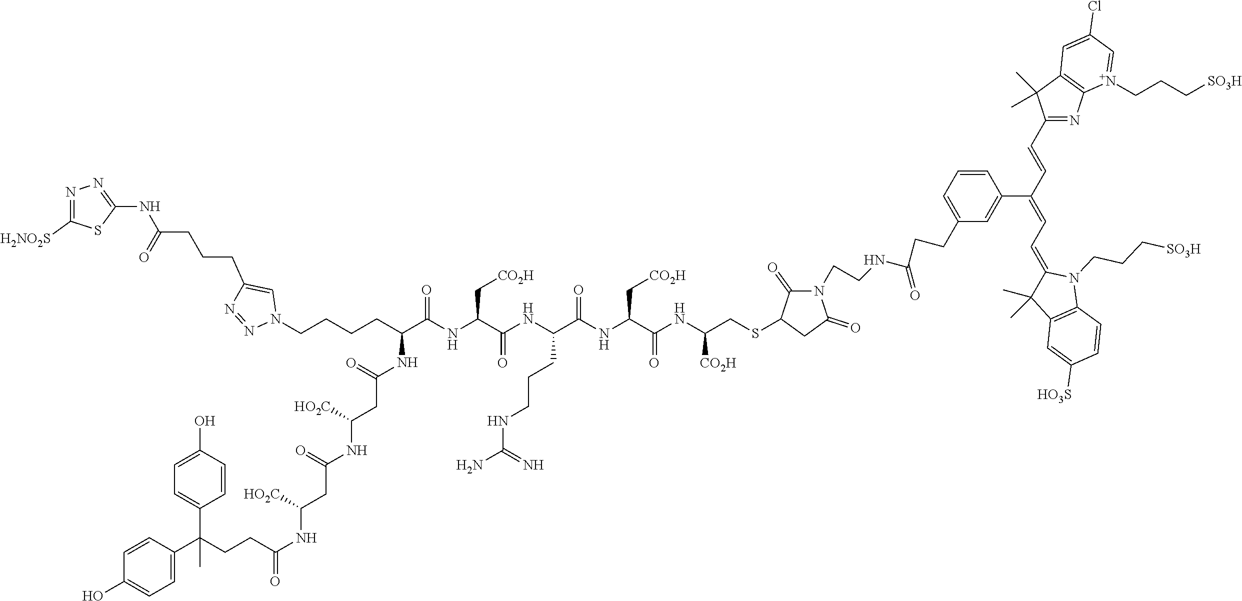 [SMILES: CC1(C)C(/C=C/C(=C/C=C2\N(CCCS(=O)(=O)O)c3ccc(S(=O)(=O)O)cc3C2(C)C)c2cccc(CCC(=O)NCCN3C(=O)CC(SC[C@H](NC(=O)[C@H](CC(=O)O)NC(=O)[C@H](CCCNC(=N)N)NC(=O)[C@H](CC(=O)O)NC(=O)[C@H](CCCCn4cc(CCCC(=O)Nc5nnc(S(N)(=O)=O)s5)nn4)NC(=O)C[C@H](NC(=O)C[C@H](NC(=O)CCC(C)(c4ccc(O)cc4)c4ccc(O)cc4)C(=O)O)C(=O)O)C(=O)O)C3=O)c2)=Nc2c1cc(Cl)c[n+]2CCCS(=O)(=O)O]

Chemical Formula: C₁₀₁H₁₂₆ClN₂₂O₃₄S₆

### Molecular Weight: 2420.05

Compound 1 (2.5 mg, 1.70 μmol, 1.6 eq) was dissolved in degassed TBS (pH 7.6; 800 μl). Commercially available Maleimidocaproyl-IRDy680RD (1.1 mg, 1.10 μmol, 1.0 eq) was added as a DMF solution (200 μl) and the mixture was stirred at room temperature. After UPLC-MS indicated completion, the solvents were removed under vacuum and the crude was diluted in a 1:1 H₂O/MeCN mixture (1 ml). The mixture was purified by RP-HPLC (Synergi RP Polar, 5% MeCN in 0.1% aq. TFA to 80% over 20 min). Pure fractions were collected and lyophilized overnight to achieve 11 as a purple solid (1.3 mg; 0.85 μmol; 48.1% yield).

MS (ESI) m/z calcd. for [C₁₀₁H₁₂₆ClN₂₂O₃₄S₆]²⁺: 1208.8410 [M]²⁺, found: 1208.8380.

### Synthesis of the NH₂*-IRDye680RD (Negative Control; Compound 12)

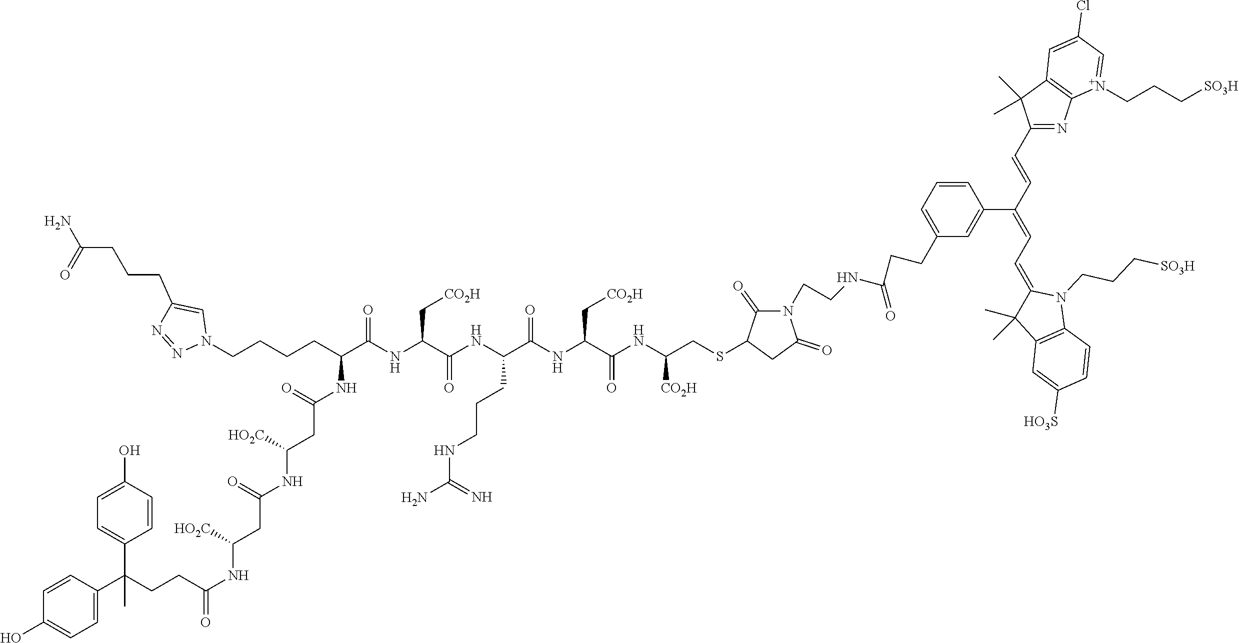 [SMILES: CC1(C)C(/C=C/C(=C/C=C2\N(CCCS(=O)(=O)O)c3ccc(S(=O)(=O)O)cc3C2(C)C)c2cccc(CCC(=O)NCCN3C(=O)CC(SC[C@H](NC(=O)[C@H](CC(=O)O)NC(=O)[C@H](CCCNC(=N)N)NC(=O)[C@H](CC(=O)O)NC(=O)[C@H](CCCCn4cc(CCCC(N)=O)nn4)NC(=O)C[C@H](NC(=O)C[C@H](NC(=O)CCC(C)(c4ccc(O)cc4)c4ccc(O)cc4)C(=O)O)C(=O)O)C(=O)O)C3=O)c2)=Nc2c1cc(Cl)c[n+]2CCCS(=O)(=O)O]

Chemical Formula: C₉₉H₁₂₅ClN₁₉O₃₂S₄

### Molecular Weight: 2256.88

Compound 5 (1.7 mg, 1.30 μmol, 1.6 eq) was dissolved in degassed TBS (pH 7.6; 800 μl). Commercially available Maleimidocaproyl-IRDy680RD (1.1 mg, 1.10 μmol, 1.0 eq) was added as a DMF solution (200 μl) and the mixture was stirred at room temperature. After UPLC-MS indicated completion, the solvents were removed under vacuum and the crude was diluted in a 1:1 H₂O/MeCN mixture (1 ml). The mixture was purified by RP-HPLC (Synergi RP Polar, 5% MeCN in 0.1% aq. TFA to 80% over 20 min). Pure fractions were collected and lyophilized overnight to achieve 12 as a purple solid (1.5 mg; 0.66 μmol; 60.1% yield).

MS (ESI) m/z calcd. for [C₉₉H₁₂₅ClN₁₉O₃₂S₄]²⁹⁺: 1217.3655 [M]²⁺, found: 1217.3931.
Radiolabelling and Quantitative Biodistribution Study of AAZ*-⁹⁹mTc (Compound 8) and of AAZ-⁹⁹mTc (Compound 9) in the SKRC-52 Model (and Negative Control, Compound 10)

Radiolabeling procedures with technetium-99m were performed following described procedures (Krall N, Pretto F, Mattarella M, Muller C, Neri D. A technetium 99m-labeled ligand of carbonic anhydrase IX selectively targets renal cell carcinoma in vivo. J Nucl Med 2016; and Cazzamalli S, Dal Corso A, Neri D. Acetazolamide serves as selective delivery vehicle for dipeptide-linked drugs to renal cell carcinoma. Mol Cancer Ther 2016). Briefly, compounds 8, 9 and 10 (60 nmol) in TBS pH 7.4 (50 μl) were separately mixed with SnCl₂ (Sigma Aldrich, 200 μg) and sodium glucoheptonate (TCI, 20 mg) in H₂O (150 μl). Tris-buffered saline at pH 7.4 (600 μl) was added and the resulting solution degassed for 5 min by bubbling with nitrogen gas. The eluate from a ^{99m}Tc-generator (200 μl, ca. 200 MBq, Mallinckrodt) was added and the reaction mixture heated to 90° C. for 20 min After cooling to room temperature, analytic aliquots were analyzed by RP-HPLC (XTerra C18, 5% MeCN in 0.1% aq. TFA to 80% over 20 min on a Merck-Hitachi D-7000 HPLC system equipped with a Raytest Gabi Star radiodetector) to determine radiolabelling yields. ⁹⁹mTc incorporations >95% were routinely achieved.

SKRC-52 xenografted tumors were implanted into female BALB/c nu/nu mice (Janvier) as described above, and allowed to grow for three weeks to an average volume of 250 mm³. Mice were randomized (n=3 per group) and injected intravenously with radiolabelled preparations of compounds 8, 9 or 10 (1-3 MBq, 30 nmol/Kg). Mice were sacrificed 6 hours after the injection by CO₂ asphyxiation and organs extracted, weighted and radioactivity measured with a Packard Cobra γ-counter. Values are expressed as % ID/g ±SD (FIG. 10B).

### In Vivo Imaging System (IVIS): Near Infrared Fluorescence Imaging Evaluation

Female BALB/c nude mice bearing subcutaneous SKRC-52 tumors were injected intravenously with AAZ* labeled with the near infrared dye moiety IRDye680RD (AAZ*-IRDye680RD; compound 11; 250 nmol/Kg), dissolved in sterile PBS (100 μl), or, alternatively, with negative compound 12 (250 nmol/Kg; dissolved in sterile PBS, 100 μl). Mice were anesthetized with isoflurane and fluorescence images acquired on an IVIS Spectrum imaging system (Xenogen, exposure 1 s, binning factor 8, excitation at 675 nm, emission filter at 720 nm, f number 2, field of view 13.1). Images were taken before the injection and after 5 min, 1 hour, 3 hours and 6 hours. Food and water were given ad libitum during that period. Mice were sacrificed after the last picture by cervical dislocation.

### Results

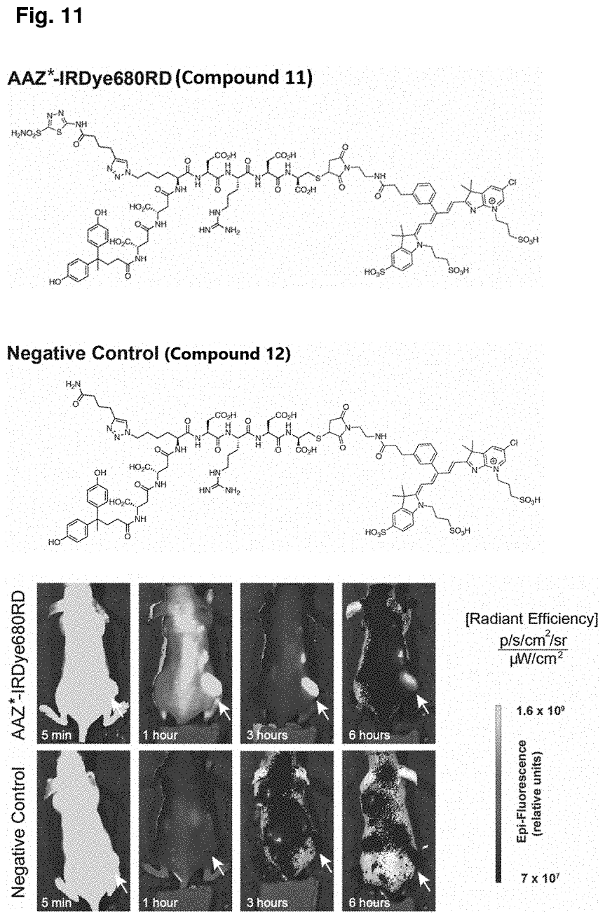

The compound 8 accumulated with 40% ID/g in the tumor mass six hours after intravenous administration (with a tumor to blood ratio of 80:1), while the similar compound 9 featuring acetazolamide (AAZ) as CAIX binder exhibited a 18% ID/g at the same time point (FIG. 10B). Similar results were obtained with AAZ*-IRDye680RD (compound 11) that selectively accumulated at the site of SKRC-52 tumors in vivo, as demonstrated by near infrared fluorescence imaging evaluation (FIG. 11). No preferential tumor uptake was observed in the case of the negative controls compound 10 (FIG. 10B) and 12 (FIG. 11).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

Sequence Listing

Amino acid sequence of the L19-VH (SEQ ID NO: 1)
EVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFP
YFDYWGQGTLVTVSS
Linker linking the L19-VH and L19-VL (SEQ ID NO: 2)
GDGSSGGSGGAS
Amino acid sequence of the L19-VL (SEQ ID NO: 3)
EIVLTQSPGTLSLSPGERATLSCRASQSVSSSFLAWYQQKPGQAPRLLIYYA
SSRATGIPDRFSGSGSGTDFTLTISRLEPEDFAVYYCQQTGRIPPTFGQGTKV
EIK
Amino acid sequence of the L19 scFv (SEQ ID NO: 4)
The VH and VL domain CDRs of the L19 antibody are underlined.
The linker sequence is shown in bold and underlined.
EVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFP
YFDYWGQGTLVTVSSGDGSSGGSGGASEIVLTQSPGTLSLSPGERATLSCR
ASQSVSSSFLAWYQQKPGQAPRLLIYYASSRATGIPDRFSGSGSGTDFTLTI
SRLEPEDFAVYYCQQTGRIPPTFGQGTKVEIK
Amino acid sequences of the L19 CDRs
L19 CDR1 VH-SFSMS (SEQ ID NO: 5)
L19 CDR2 VH-SISGSSGTTYYADSVKG (SEQ ID NO: 6)
L19 CDR3 VH-PFPYFDY (SEQ ID NO: 7)
L19 CDR1 VL-RASQSVSSSFLA (SEQ ID NO: 8)
L19 CDR2 VL-YASSRAT (SEQ ID NO: 9)
L19 CDR3 VL-QQTGRIPPT (SEQ ID NO: 10)
Amino acid sequence of the F8 VH domain (SEQ ID NO: 11)
EVQLLESGGGLVQPGGSLRLSCAASGFTFSLFTMSWVRQAPGKGLEWVSA
ISGSGGSTYYADSVKGRFTISRDNSKNTLYLQMNSLRAEDTAVYYCAKST
HLYLFDYWGQGTLVTVSS
Amino acid sequence of the diabody linker linking the F8 VH
domain to the F8 VL domain (SEQ ID NO: 12)
GGSGG
Amino acid sequence of the F8 VL domain (SEQ ID NO: 13)
EIVLTQSPGTLSLSPGERATLSCRASQSVSMPFLAWYQQKPGQAPRLLIYG
ASSRATGIPDRFSGSGSGTDFTLTISRLEPEDFAVYYCQQMRGRPPTFGQGT
KVEIK
Amino acid sequence of the F8 diabody (SEQ ID NO: 14)
The VH and VL domain CDRs of the F8 antibody are underlined.
The linker sequence is shown in bold and underlined.
EVQLLESGGGLVQPGGSLRLSCAASGFTFSLFTMSWVRQAPGKGLEWVSA
ISGSGGSTYYADSVKGRFTISRDNSKNTLYLQMNSLRAEDTAVYYCAKST
HLYLFDYWGQGTLVTVSSGGSGGEIVLTQSPGTLSLSPGERATLSCRAQS
VSMPFLAWYQQKPGQAPRLLIYGASSRATGIPDRFSGSGSGTDFTLTISRLE
PEDFAVYYCQQMRGRPPTFGQGTKVEIK
Amino acid sequences of the F8 CDRs
F8 CDR1 VH-LFT (SEQ ID NO: 15)
F8 CDR2 VH-SGSGGS (SEQ ID NO: 16)
F8 CDR3 VH-STHLYL (SEQ ID NO: 17)
F8 CDR1 VL-MPF (SEQ ID NO: 18)
F8 CDR2 VL-GASSRAT (SEQ ID NO: 19)
F8 CDR3 VL-MRGRPP (SEQ ID NO: 20)
Amino acid sequence of the human IL2 (SEQ ID NO: 21)
APTSSSTKKTQLQLEHLLLDLQMILNGINNYKNPKLTRMLTFKFYMPKKAT
ELKHLQCLEEELKPLEEVLNLAQSKNFHLRPRDLISNINVIVLELKGSETTF
MCEYADETATIVEFLNRWITFCQSIISTLT
Amino acid sequence of the L19-IL2 immunocytokine (SEQ ID NO: 22)
L19(scFv)-IL2: the linker linking L19 to IL2 is shown in italics
and underlined and IL2 is shown in bold.
EVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFP
YFDYWGQGTLVTVSSGDGSSGGSGGASEIVLTQSPGTLSLSPGERATLSCR
ASQSVSSSFLAWYQQKPGQAPRLLIYYASSRATGIPDRFSGSGSGTDFTLTI
SRLEPEDFAVYYCQQTGRIPPTFGQGTKVEIKEFSSSSGSSSSGSSSSGAPTSS
STKKTQLQLEHLLLDLQMILNGINNYKNPKLTRMLTEKEYMPKKATE
LKHLQCLEEELKPLEEVLNLAQSKNFHLRPRDLISNINVIVLELKGSET
TFMCEYADETATIVEFLNRWITFCQSIISTLT
Amino acid sequence of the linker linking the L19 scFv and
IL2 (SEQ ID NO: 23)
EFSSSSGSSSSGSSSSG
Amino acid sequence of the F8-IL2 immunocytokine (SEQ ID NO: 24)
F8(diabody)-IL2: the linker linking F8 to IL2 is shown in italics
and underlined and IL2 is shown in bold.
EVQLLESGGGLVQPGGSLRLSCAASGFTFSLFTMSWVRQAPGKGLEWVSA
ISGSGGSTYYADSVKGRFTISRDNSKNTLYLQMNSLRAEDTAVYYCAKST
HLYLFDYWGQGTLVTVSSGGSGGEIVLTQSPGTLSLSPGERATLSCRASQS
VSMPFLAWYQQKPGQAPRLLIYGASSRATGIPDRFSGSGSGTDFTLTISRLE
PEDFAVYYCQQMRGRPPTFGQGTKVEIKSSSSGSSSSGSSSSGAPTSSSTKKT
QLQLEHLLLDLQMILNGINNYKNPKLTRMLTEKEYMPKKATELKHLQ
CLEEELKPLEEVLNLAQSKNFHLRPRDLISNINVIVLELKGSETTFMCE
YADETATIVEFLNRWITFCQSIISTLT
Amin acid sequence of the linker linking the F8 diabody and
IL2 (SEQ ID NO: 25)
SSSSGSSSSGSSSSG
Amino acid sequence of peptide spacer
Asp-Arg-Asp-Cys (SEQ ID NO: 26)

### REFERENCES

1. Nikolaus Krall et al., “Small Targeted Cytotoxics: Current State and Promises from DNA-Encoded Chemical Libraries,” Angew Chem Int Edit, vol. 52, No. 5, January 2013, pages 1384-1402.
2. WO 2004/048544 A2.
3. US 2006/057068 A1.
4. WO 2013/167994 A1.
5. Nikolaus Krall et al., “A Small-Molecule Drug Conjugate for the Treatment of Carbonic Anhydrase IX Expressing Tumors,” Angew Chem Int Edit, vol. 53, No. 16, March 2014, pages 4231-4235.
6. Moreno Wichert et al., “Dual-display of small molecules enables the discovery of ligand pairs and facilitates affinity D6 maturation,” Nat Chem, vol. 7, no. 3, January 2015, pages 241-249.
7. Gualberto A., “Brentuximab Vedotin (SGN-35), an antibody-drug conjugate for the treatment of CD30-positive malignancies,” Expert Opin Inv Drug, vol. 21, no. 2, February 2012, pages 205-216.
8. Beck A. and Reichert J. M., “Antibody-drug conjugates: present and future,” MAbs, vol. 6, no. 1, January-February 2014, pages 15-17.
9. Van der Veldt et al., “Biodistribution and radiation dosimetry of 11C-labelled docetaxel in cancer patients,” Eur J Nucl Med Mol I, vol. 37, no. 10, October 2010, pages 1950-1958.
10. Van der Veldt et al., “Toward prediction of efficacy of chemotherapy: a proof of concept study in lung cancer patients using [¹¹C]docetaxel and positron emission tomography,” Clin Cancer Res, vol. 19, no. 15, August 2013, pages 4163-4173.
11. Cazzamalli Samuele et al., “Acetazolamide serves as selective delivery vehicle for dipeptide-linked drugs to renal cell carcinoma” Mol Cancer Ther, vol. 15, no. 12, September 2016, pages 2926-2935.

1. A composition comprising
(i) an immunocytokine and
(ii) a small molecule drug conjugate (SMDC),
wherein
the immunocytokine comprises an antibody or antibody fragment conjugated to a cytokine, and
the small molecule comprises a moiety capable of binding to a tumor-associated target.

2. The composition of claim 1 wherein the moiety is specific for carbonic anhydrase IX (CAIX),
and/or
the immunocytokine comprises:
(a) an antibody targeting the ED-B or ED-A domain of fibronectin, and
(b) interleukin-2.

3. (canceled)

4. The composition of claim 2, wherein the SMDC comprises:
(c) a ligand moiety capable of binding to CAIX,
(d) a linker, and
(e) a cytotoxic drug.

5. (canceled)

6. The composition of claim 4, wherein the moiety capable of binding to CAIX has a terminal sulfonamide (—SO₂NH₂), sulfamate (—OSO₂NH₂) or sulfamide (—NHSO₂NH₂) group.

7. (canceled)

8. The composition of claim 6, wherein the moiety capable of binding to CAIX is 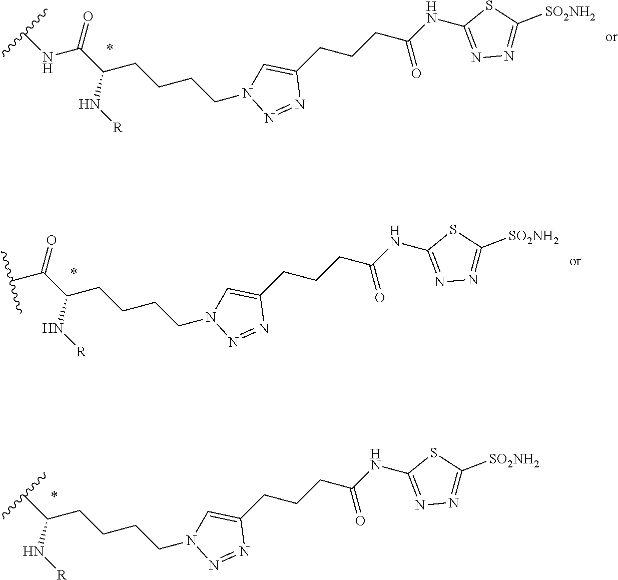
wherein R is: 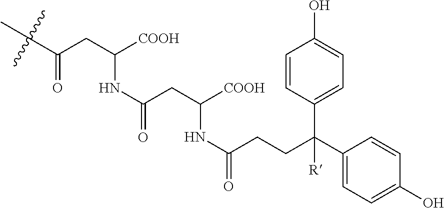
wherein R′ is H or C1-C7 alkyl, C1-C7 alkenyl, or C1-C7 heteroalkyl, optionally substituted with one, two or three substituents, and preferably R′ is methyl.

9. (canceled)

10. The composition of claim 1, wherein the drug is a cytotoxic drug.

11. The composition of claim 10, wherein the cytotoxic drug is selected from the group consisting of dolastatin, a dolastatin analogue, and a dolastatin derivative.

12. (canceled)

13. The composition of claim 4, wherein the drug is attached to the ligand by a cleavable linker.

14. (canceled)

15. The composition of claim 13, wherein the linker is selected from valine-citrulline or valine-alanine.

16. (canceled)

17. The composition of claim 1, wherein the cytokine is an interleukin selected from the group consisting of IL-2, IL-12 and TNF.

18. The composition of claim 1, wherein the antibody fragment is selected from the group consisting of ScFv, diabody and SIP.

19. The composition of claim 13, wherein the cleavable linker comprises
(1) a disulfide bond;
(2) an amide linkage; or
(3) an ester linkage.

20. The composition of claim 19, wherein, when the cleavable linker comprises
(1) a disulfide bond, the cleavage agent comprises a reducing agent;
(2) an amide linkage, the cleavage agent comprises a hydrolase; or
(3) an ester linkage, the cleavage agent comprises a hydrolase.

21. The composition of claim 2, wherein the SMDC is compound 2: 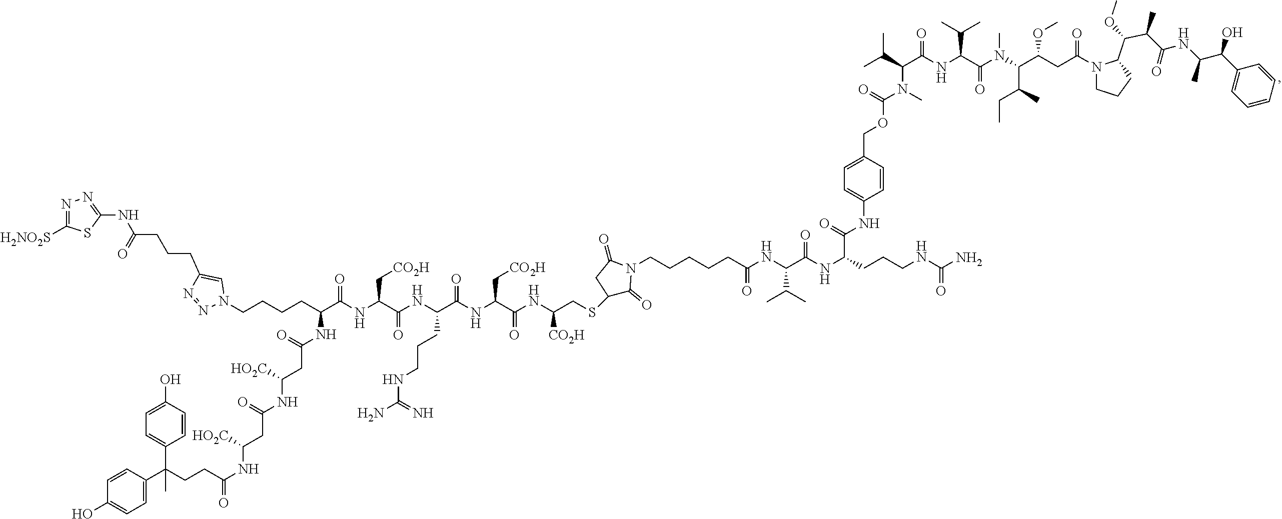 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC[C@H](NC(=O)[C@H](CC(=O)O)NC(=O)[C@H](CCCNC(=N)N)NC(=O)[C@H](CC(=O)O)NC(=O)[C@H](CCCCn3cc(CCCC(=O)Nc4nnc(S(N)(=O)=O)s4)nn3)NC(=O)C[C@H](NC(=O)C[C@H](NC(=O)CCC(C)(c3ccc(O)cc3)c3ccc(O)cc3)C(=O)O)C(=O)O)C(=O)O)C2=O)C(C)C)cc1)C(C)C]
(acetazolamide-ValCit-(mono methyl Auristatin E; “AAZ*-ValCit-MMAE”).

22. A pharmaceutical composition comprising the composition of claim 1 and a pharmaceutically-acceptable excipient.

23. A method of treating a neoplastic disease comprising administering to a patient in need thereof effective amounts of a composition of claim 1.

24. The method of claim 23, wherein the immunocytokine and SMDC are administered concurrently, or are administered sequentially in either order, optionally in repeated cycles of administration.

25. (canceled)

26. The method of claim 23, wherein the neoplastic disease is kidney cancer or a colorectal cancer.

27. The method of claim 23, wherein, when the cleavable linker comprises
(1) a disulfide bond, the cleavage agent comprises a reducing agent selected from cysteine, N-acetylcysteine, or ordithiothreitol; or
(2) an amide linkage, the cleavage agent comprises a hydrolase that is a protease.

28. The composition of claim 2, wherein the immunocytokine comprises an antibody targeting the ED-B or ED-A domain of fibronectin.

29. (canceled)

30. The composition of claim 28, wherein the antibody targeting the ED-B domain of fibronectin is L19, or the antibody targeting the ED-A domain of fibronectin in F8.

31. (canceled)

32. (canceled)

33. (canceled)

35. (canceled)

36. (canceled)
